(19) United States
(12) Patent Application Publication
(10) Pub. No.: US 2024/0158505 A1
(43) Pub. Date: May 16, 2024

(54) B7-H3 ANTIBODY OR ANTIGEN-BINDING FRAGMENT THEREOF, AND USE THEREOF

(71) Applicant: THERANOTICS CO., LTD., Gyeonggi-do (KR)

(72) Inventors: Byung Hun JUNG, Seoul (KR); Jung Wook LEE, Seoul (KR); Dong Woon PARK, Gyeonggi-do (KR); Jung Hee KANG, Seoul (KR); Jung Eun LEE, Gyeonggi-do (KR)

(21) Appl. No.: 18/284,142

(22) PCT Filed: Mar. 24, 2022
(86) PCT No.: PCT/KR2022/004114

§ 371 Date: Sep. 26, 2023

(30) Foreign Application Priority Data
Mar. 26, 2021 (KR) 10-2021-0039913
Mar. 24, 2022 (KR) 10-2022-0036430

(51) Int. Cl. C07K 16/28; A61K 39/00; A61P 35/00

(52) U.S. Cl. CPC C07K 16/2827; A61P 35/00; A61K 2039/505

(57) ABSTRACT

A B7-H3 antibody or antigen-binding fragment thereof includes a heavy chain variable region including heavy chain complementarity determining regions (HCDRs) and a light chain variable region including light chain complementarity determining regions (LCDRs). The B7-H3 antibody has a predetermined complementarity determining region, thereby specifically binding to a B7-H3 antigen, and being internalized into cells, and being usable as an immune checkpoint inhibitor for various diseases.

### CROSS REFERENCE TO RELATED APPLICATIONS AND CLAIM OF PRIORITY

This application claims benefit under 35 U.S.C. 119, 120, 121, or 365 (c), and is a National Stage entry from International Application No. PCT/KR2022/004114, filed Mar. 24, 2022, which claims priority to the benefit of Korean Patent Application Nos. 10-2021-0039913 filed on Mar. 26, 2021, and 10-2022-0036430 filed on Mar. 24, 2022, in the Korean Intellectual Property Office, the entire contents of which are incorporated herein by reference.

### BACKGROUND

### 1. Technical Field

The present invention relates to a novel B7-H3 antibody.

### 2. Background Art

B7 homology 3 protein (B7-H3) (also referred to as CD276 and B7RP-2, which are collectively referred to as B7-H3 herein) is a type I transmembrane glycoprotein that belongs to the immunoglobulin superfamily.

Human B7-H3 includes a putative signal peptide, V-like and C-like Ig domains, a transmembrane region and a cytoplasmic domain. Exon duplication in humans leads to expression of two B7-H3 isoforms having any one of an IgV-IgC-IgV-IgC-like domain including several conserved cysteine residues (4IgB7-H3 isoform) or a single IgV-IgC-like domain (2IgB7-H3 isoform). Predominant B7-H3 isoform in human tissues and cell lines is a 4IgB7-H3 isoform.

It has been reported that B7-H3 has both co-stimulatory and co-inhibitory signaling functions.

B7-H3 is not constitutively expressed on many immune cells (e.g., natural killer (NK) cells, T-cells, and antigen-presenting cells (APCs)), but its expression can be induced on these cells.

In addition, expression of B7-H3 is not restricted to the immune cells. B7-H3 transcripts are expressed in a wide spectrum of human tissues including colon, heart, liver, placenta, prostate, small intestine, testis and uterus; and in osteoblasts, fibroblasts, epithelial cells, and other cells of non-lymphoid lineage, which potentially exhibit immunological and non-immunological functions. However, protein expression in normal tissues is typically maintained at a low level, thus post-transcriptional regulation may be applied thereto.

### SUMMARY

An object of the present invention is to provide a novel B7-H3 antibody or antigen-binding fragment thereof.

Another object of the present invention is to provide a medical use (such as a pharmaceutical composition, treatment method, etc.) of B7-H3 antibody or antigen-binding fragment thereof.

1. A B7-H3 antibody or antigen-binding fragment thereof including a heavy chain variable region which includes HCDRs below and a light chain variable region which includes LCDRs below:
(a) HCDRs of SEQ ID NOs: 1, 10 and 19 and LCDRs of SEQ ID NOs: 28, 37 and 45;
(b) HCDRs of SEQ ID NOs: 2, 11 and 20 and LCDRs of SEQ ID NOs: 29, 38 and 46;
(c) HCDRs of SEQ ID NOs: 3, 12 and 21 and LCDRs of SEQ ID NOs: 30, 39 and 47;
(d) HCDRs of SEQ ID NOs: 4, 13 and 22 and LCDRs of SEQ ID NOs: 31, 40 and 48;
(e) HCDRs of SEQ ID NOs: 5, 14 and 23 and LCDRs of SEQ ID NOs: 32, 41 and 49;
(f) HCDRs of SEQ ID NOs: 6, 15 and 24 and LCDRs of SEQ ID NOs: 33, 42 and 50;
(g) HCDRs of SEQ ID NOs: 7, 16 and 25 and LCDRs of SEQ ID NOs: 34, 43 and 51;
(h) HCDRs of SEQ ID NOs: 8, 17 and 26 and LCDRs of SEQ ID NOs: 35, 44 and 52; or
(i) HCDRs of SEQ ID NOs: 9, 18 and 27 and LCDRs of SEQ ID NOs: 36, 42 and 53.

2. The B7-H3 antibody or antigen-binding fragment thereof according to the above 1, wherein the heavy chain variable region includes any one framework sequence selected from the group consisting of HFRs below:
(hf1) HFRs of SEQ ID NOs: 54, 63, 68 and 334;
(hf2) HFRs of SEQ ID NOs: 55, 63, 69 and 334;
(hf3) HFRs of SEQ ID NOs: 56, 64, 70 and 334;
(hf4) HFRs of SEQ ID NOs: 56, 64, 71 and 334;
(hf5) HFRs of SEQ ID NOs: 57, 64, 70 and 334;
(hf6) HFRs of SEQ ID NOs: 58, 64, 72 and 334;
(hf7) HFRs of SEQ ID NOs: 59, 65, 73 and 334;
(hf8) HFRs of SEQ ID NOs: 60, 65, 73 and 334;
(hf9) HFRs of SEQ ID NOs: 61, 66, 74 and 334; and
(hf10) HFRs of SEQ ID NOs: 62, 67, 75 and 334.

3. The B7-H3 antibody or antigen-binding fragment thereof according to the above 1, wherein the light chain variable region includes any one framework sequence selected from the group consisting of LFRs below:
(lf1) LFRs of SEQ ID NOs: 76, 82, 86 and 335;
(lf2) LFRs of SEQ ID NOs: 77, 82, 87 and 335;
(lf3) LFRs of SEQ ID NOs: 78, 83, 88 and 335;
(lf4) LFRs of SEQ ID NOs: 79, 84, 89 and 335;
(lf5) LFRs of SEQ ID NOs: 80, 84, 90 and 335;
(lf6) LFRs of SEQ ID NOs: 80, 84, 91 and 335;
(lf7) LFRs of SEQ ID NOs: 81, 85, 92 and 335;
(lf8) LFRs of SEQ ID NOs: 93, 98, 101 and 336;
(lf9) LFRs of SEQ ID NOs: 93, 98, 102 and 336;
(lf10) LFRs of SEQ ID NOs: 93, 98, 103 and 336;
(lf11) LFRs of SEQ ID NOs: 93, 98, 104 and 336;
(lf12) LFRs of SEQ ID NOs: 94, 98, 105 and 336;
(lf13) LFRs of SEQ ID NOs: 95, 99, 106 and 336;
(lf14) LFRs of SEQ ID NOs: 96, 99, 107 and 336; and
(lf15) LFRs of SEQ ID NOs: 97, 100, 108 and 336.

4. The B7-H3 antibody or antigen-binding fragment thereof according to the above 1, wherein the heavy chain variable region is any one selected from the group consisting of SEQ ID NOs: 127, 128, 129, 130, 131, 132, 135, 142 and 152.

5. The B7-H3 antibody or antigen-binding fragment thereof according to the above 1, wherein the light chain variable region is any one selected from the group consisting of SEQ ID NOs: 211, 221, 223, 224, 225, 231, 307, 309 and 317.

6. A gene encoding the B7-H3 antibody or antigen-binding fragment thereof of any one of the above 1 to 5.

7. A cell including a vector introduced therein, in which the gene of the above 6 is inserted.

8. A method for preparing a B7-H3 antibody or antigen-binding fragment thereof including culturing the cell of the above 7.

9. A pharmaceutical composition for treating or preventing cancer including the B7-H3 antibody or antigen-binding fragment thereof of any one of the above 1 to 5.

10. The pharmaceutical composition according to the above 9, wherein the cancer is any one selected from the group consisting of lung cancer, breast cancer, ovarian cancer, uterine cancer, cervical cancer, glioma, neuroblastoma, prostate cancer, pancreatic cancer, colorectal cancer, colon cancer, head and neck cancer, leukemia, lymphoma, renal cancer, bladder cancer, gastric cancer, liver cancer, skin cancer, brain tumor, cerebrospinal cancer, adrenal tumor, melanoma, sarcoma, multiple myeloma, pancreatic neuroendocrine neoplasm, peripheral nerve sheath tumor and small cell tumor.

11. A method for treating cancer including administering the B7-H3 antibody or antigen-binding fragment thereof of any one of the above 1 to 5, or a gene encoding the same, to a subject.

12. The method according to the above 11, wherein the cancer is any one selected from the group consisting of lung cancer, breast cancer, ovarian cancer, uterine cancer, cervical cancer, glioma, neuroblastoma, prostate cancer, pancreatic cancer, colorectal cancer, colon cancer, head and neck cancer, leukemia, lymphoma, renal cancer, bladder cancer, gastric cancer, liver cancer, skin cancer, brain tumor, cerebrospinal cancer, adrenal tumor, melanoma, sarcoma, multiple myeloma, pancreatic neuroendocrine neoplasm, peripheral nerve sheath tumor and small cell tumor.

13. The B7-H3 antibody or antigen-binding fragment thereof of any one of the above 1 to 5, wherein B7-H3 antibody or antigen-binding fragment thereof is used as a medicament for treatment of cancer.

14. The B7-H3 antibody or antigen-binding fragment thereof according to the above 13, wherein the medicament is an anticancer drug.

15. The B7-H3 antibody or antigen-binding fragment thereof according to the above 13, wherein the cancer is any one selected from the group consisting of lung cancer, breast cancer, ovarian cancer, uterine cancer, cervical cancer, glioma, neuroblastoma, prostate cancer, pancreatic cancer, colorectal cancer, colon cancer, head and neck cancer, leukemia, lymphoma, renal cancer, bladder cancer, gastric cancer, liver cancer, skin cancer, brain tumor, cerebrospinal cancer, adrenal tumor, melanoma, sarcoma, multiple myeloma, pancreatic neuroendocrine neoplasm, peripheral nerve sheath tumor and small cell tumor.

The B7-H3 antibody or antigen-binding fragment thereof of the present invention specifically binds to B7-H3.

The B7-H3 antibody or antigen-binding fragment thereof of the present invention may allow B7-H3 to be introduced into cells.

The B7-H3 antibody or antigen-binding fragment thereof of the present invention can be used as an immune checkpoint inhibitor.

The B7-H3 antibody or antigen-binding fragment thereof, or the gene encoding the same of the present invention may be administered to a subject to treat diseases.

The B7-H3 antibody or antigen-binding fragment thereof, or the gene encoding the antibody of the present invention may be administered in combination with an anticancer agent having a different pharmacological mechanism.

### BRIEF DESCRIPTION OF DRAWINGS

FIG. 1 shows binding affinity and EC₅₀ values according to the concentrations of #1 to #9 antibodies to B7-H3.

FIG. 2 shows binding affinity according to the concentrations of #1 to #9 antibodies to MCF-7 cell line.

FIG. 3 shows binding affinity according to the concentrations of #1 to #9 antibodies to RKO cell line.

FIG. 4 shows binding affinity according to the concentrations of #1 to #9 antibodies to RKO cells (RKO/B7H3) in which B7-H3 protein is overexpressed.

FIG. 5 illustrates measurement of internalization of the antibodies after treating the MCF-7 cell line with respective pHAb amine-labeled antibodies.

FIG. 6 illustrates measurement of the internalization of the antibodies after treating the RKO cell line and the RKO/B7H3 cell line with respective pHAb amine-labeled secondary antibodies.

FIGS. 7 and 8 show invasion assay results of RKO, RKO/B7H3, and RKO/B7H3 treated with respective antibodies. Specifically, FIG. 7 are images taken of the degree of invasion using a microscope, and FIG. 8 illustrates calculation of the percentage of invaded cells using Image J.

FIGS. 9 and 10 show migration assay results of RKO, RKO/B7H3, and RKO/B7H3 treated with respective antibodies. Specifically, FIG. 9 are images taken of the degree of migration using a microscope, and FIG. 10 illustrates calculation of the percentage of OD values measured by extracting colors of cells stained with crystal violet.

FIG. 11 illustrates classification of #1 to #9 antibodies according to common epitopes.

FIG. 12 shows TGFβ secretion assay results after treating the RKO/B7H3 cell line with #1 to #9 antibodies.

FIG. 13 shows a standard dilution process in the TGFβ secretion assay.

FIG. 14 shows changes in tumor volume after antibody administration in mice implanted with cell line CT26-TN cells overexpressing the B7-H3 in colorectal cancer cell line CT26.

Wherein, G1 (vehicle) and G2 (IgG) represent the negative controls, G3 (#5) represents a #5 antibody administration group, and G4 (#5)+Co represents a #5 antibody and anti-PD-1 antibody combined administration group.

FIG. 15 shows changes in TGFβ concentration in mouse serum by #5 antibody. Wherein, Vehicle and IgG represent the negative controls, #5 represents the #5 antibody administration group, and #5+Co represents the #5 antibody and anti-PD-1 antibody combined administration group. *: p value <0.5 (compared with vehicle group)

FIG. 16 shows the number of immune cells in the tumor after antibody treatment. Wherein, G1 (vehicle) and G2 (IgG) represent the negative controls, G3 (#5) represents the #5 antibody administration group, and G4 (#5)+Co represents the #5 antibody and anti-PD-1 antibody combined administration group.

### DETAILED DESCRIPTION

The present invention relates to a B7-H3 antibody or antigen-binding fragment thereof.

In the present invention, the antigen-binding fragment of the B7-H3 antibody refers to one or more fragments of the antibody that maintain the ability to specifically bind to the B7-H3.

The antibody may be of any type (e.g., IgG, IgE, IgM, IgD, IgA and IgY), class (e.g., IgG1, IgG2, IgG 3, IgG4, IgA and IgA2, etc.) or subclass.

The antigen-binding fragment includes: (i) a Fab fragment which is a monovalent fragment consisting of VH, VL, CH1 and CL domains; (ii) a F(ab′)₂ fragment which is a bivalent fragment including two Fab fragments linked by a disulfide bond in a hinge region; (iii) a Fd fragment consisting of VH and CH1 domains; (iv) an Fv fragment consisting of VL and VH domains of a single arm of an antibody; (v) a single domain or dAb fragment consisting of VH domain; (vi) an isolated complementarity determining region (CDR); and (vii) a combination of two or more isolated CDRs optionally linked by a synthetic linker.

In addition, the VL domain and the VH domain of the Fv fragment are encoded by separated genes, but they may be linked by the synthetic linker using a recombinant method so as to produce a single protein chain having a monovalent molecule (called a single chain Fv (scFv) or single chain antibody) by pairing with the VL and VH domains. This single chain antibody (scFv) is also included in the antigen-binding fragment.

The antigen-binding fragment is obtained using the conventional techniques known in the art, and functional screening of the fragment is used in the same way as for the intact antibody. Antigen binding sites may be produced by recombinant DNA technique or by enzymatic or chemical disruption of the intact immunoglobulin. The antibodies may be present as different phenotypic antibodies, for example, IgG (e.g., IgG1, IgG2, IgG3 or IgG4 subtypes), IgA1, IgA2, IgD, IgE or IgM antibodies.

The B7-H3 antibody or antigen-binding fragment thereof of the present invention includes a heavy chain variable region (VH) and a light chain variable region (VL).

The heavy chain variable region of the present invention includes heavy chain complementarity determining regions (HCDRs) below, and the light chain variable region includes light chain complementarity determining regions (LCDRs) below: (a) HCDRs of SEQ ID NOs: 1, 10 and 19 and LCDRs of SEQ ID NOs: 28, 37 and 45; (b) HCDRs of SEQ ID NOs: 2, 11 and 20 and LCDRs of SEQ ID NOs: 29, 38 and 46; (c) HCDRs of SEQ ID NOs: 3, 12 and 21 and LCDRs of SEQ ID NOs: 30, 39 and 47; (d) HCDRs of SEQ ID NOs: 4, 13 and 22 and LCDRs of SEQ ID NOs: 31, 40 and 48; (e) HCDRs of SEQ ID NOs: 5, 14 and 23 and LCDRs of SEQ ID NOs: 32, 41 and 49; (f) HCDRs of SEQ ID NOs: 6, 15 and 24 and LCDRs of SEQ ID NOs: 33, 42 and 50; (g) HCDRs of SEQ ID NOs: 7, 16 and 25 and LCDRs of SEQ ID NOs: 34, 43 and 51; (h) HCDRs of SEQ ID NOs: 8, 17 and 26 and LCDRs of SEQ ID NOs: 35, 44 and 52; or (i) HCDRs of SEQ ID NOs: 9, 18 and 27 and LCDRs of SEQ ID NOs: 36, 42 and 53.

The heavy chain complementarity determining region (HCDR) consists of HCDR1, HCDR2 and HCDR3, and the light chain complementarity determining region (LCDR) consists of LCDR1, LCDR2 and LCDR3. For example, in the above sequence (a), the amino acid sequence of SEQ ID NO: 1 is HCDR1, the amino acid sequence of SEQ ID NO: 10 is HCDR2, the amino acid sequence of SEQ ID NO: 19 is HCDR3, the amino acid sequence of SEQ ID NO: 28 is LCDR1, the amino acid sequence of SEQ ID NO: 37 is LCDR2, and the amino acid sequence of SEQ ID NO: 45 is LCDR3.

The B7-H3 antibody or antigen-binding fragment thereof of the present invention specifically binds to the B7-H3 antigen regardless of the framework sequence, as long as it includes the above-described complementarity determining region.

The heavy chain variable region and the light chain variable region of the present invention may include various framework sequences.

The heavy chain variable region of the present invention may include, for example, any one sequence selected from the group consisting of heavy chain framework sequences (HFRs) below: (hf1) HFRs of SEQ ID NOs: 54, 63, 68 and 334; (hf2) HFRs of SEQ ID NOs: 55, 63, 69 and 334; (hf3) HFRs of SEQ ID NOs: 56, 64, 70 and 334; (hf4) HFRs of SEQ ID NOs: 56, 64, 71 and 334; (hf5) HFRs of SEQ ID NOs: 57, 64, 70 and 334; (hf6) HFRs of SEQ ID NOs: 58, 64, 72 and 334; (hf7) HFRs of SEQ ID NOs: 59, 65, 73 and 334; (hf8) HFRs of SEQ ID NOs: 60, 65, 73 and 334; (hf9) HFRs of SEQ ID NOs: 61, 66, 74 and 334; and (hf10) HFRs of SEQ ID NOs: 62, 67, 75 and 334.

The light chain variable region of the present invention may include, for example, any one sequence selected from the group consisting of light chain framework sequences (LFRs) below: (lf1) LFRs of SEQ ID NOs: 76, 82, 86 and 335; (lf2) LFRs of SEQ ID NOs: 77, 82, 87 and 335; (lf3) LFRs of SEQ ID NOs: 78, 83, 88 and 335; (lf4) LFRs of SEQ ID NOs: 79, 84, 89 and 335; (lf5) LFRs of SEQ ID NOs: 80, 84, 90 and 335; (lf6) LFRs of SEQ ID NOs: 80, 84, 91 and 335; (lf7) LFRs of SEQ ID NOs: 81, 85, 92 and 335; (lf8) LFRs of SEQ ID NOs: 93, 98, 101 and 336; (lf9) LFRs of SEQ ID NOs: 93, 98, 102 and 336; (lf10) LFRs of SEQ ID NOs: 93, 98, 103 and 336; (lf11) LFRs of SEQ ID NOs: 93, 98, 104 and 336; (lf12) LFRs of SEQ ID NOs: 94, 98, 105 and 336; (lf13) LFRs of SEQ ID NOs: 95, 99, 106 and 336; (lf14) LFRs of SEQ ID NOs: 96, 99, 107 and 336; and (lf15) LFRs of SEQ ID NOs: 97, 100, 108 and 336.

The heavy chain framework sequence (HFR) of the present invention consists of HFR1, HFR2, HFR3 and HFR4 and the light chain framework sequence (LFR) consists of LFR1, LFR2, LFR3 and LFR4. For example, in the above sequence (hf1), the amino acid sequence of SEQ ID NO: 54 is HFR1, the amino acid sequence of SEQ ID NO: 63 is HFR2, the amino acid sequence of SEQ ID NO: 68 is HFR3, and the amino acid sequence of SEQ ID NO: 334 is HFR4. In addition, for example, in the above sequence (lf1), the amino acid sequence of SEQ ID NO: 76 is LFR1, the amino acid sequence of SEQ ID NO: 82 is LFR2, the amino acid sequence of SEQ ID NO: 86 is LFR3, and the amino acid sequence of SEQ ID NO: 335 is LFR4.

The framework sequences (hf1 to hf10) of the heavy chain variable region and the framework sequences (lf1 to lf15) of the light chain variable region of the present invention may be arbitrarily combined.

The heavy and light chain complementarity determining region sequences and the heavy and light chain framework sequences of the present invention may be arbitrarily combined. For example, any one of the heavy and light chain complementarity determining region sequences of (a) to (i), any one of the heavy chain framework sequences of (hf1) to (hf10), and any one of the light chain framework sequences (lf1) to (lf15) may be arbitrarily combined.

The heavy chain variable region of the present invention may consist of, for example, any one amino acid sequence selected from the group consisting of SEQ ID NOs: 127, 128, 129, 130, 131, 132, 135, 142 and 152.

The light chain variable region of the present invention may consist of, for example, any one amino acid sequence selected from the group consisting of SEQ ID NOs: 211, 221, 223, 224, 225, 231, 307, 309 and 317.

The antibodies or antigen-binding fragments thereof having the complementarity determining regions of (a) to (i) of the present invention may have the same or different epitopes.

The epitope (antigenic determinant) refers to a site of the B7-H3 antigen to which an antibody or antigen-binding fragment thereof is specifically bound. The epitopes of the antibodies or antigen-binding fragments thereof having the complementarity determining regions of (a), (d), (e), (g), (h) and (i) of the present invention are identical, and the epitopes of the antibodies or antigen-binding fragments thereof having the complementarity determining regions of (b) and (c) are identical.

In one embodiment of the present invention, #1 to #9 antibodies of the B7-H3 antibody include heavy chain variable regions and light chain variable regions below: #1: a heavy chain variable region of SEQ ID NO: 127 and a light chain variable region of SEQ ID NO: 307; #2: a heavy chain variable region of SEQ ID NO: 128 and a light chain variable region of SEQ ID NO: 317; #3: a heavy chain variable region of SEQ ID NO: 129 and a light chain variable region of SEQ ID NO: 309; #4: a heavy chain variable region of SEQ ID NO: 130 and a light chain variable region of SEQ ID NO: 211; #5: a heavy chain variable region of SEQ ID NO: 131 and a light chain variable region of SEQ ID NO: 221; #6: a heavy chain variable region of SEQ ID NO: 132 and a light chain variable region of SEQ ID NO: 231; #7: a heavy chain variable region of SEQ ID NO: 142 and a light chain variable region of SEQ ID NO: 223; #8: a heavy chain variable region of SEQ ID NO: 152 and a light chain variable region of SEQ ID NO: 224; and #9: a heavy chain variable region of SEQ ID NO: 135 and a light chain variable region of SEQ ID NO: 225.

The epitopes of #1, #4, #5, #7, #8 and #9 antibodies of the present invention are identical, and the epitopes of #2 and #3 antibodies are identical.

The #1 to #9 antibodies of the present invention exhibit a strong binding force to B7-H3 and allows B7-H3 to be introduced into cells.

The present invention provides a gene encoding the above-described B7-H3 antibody or antigen-binding fragment thereof.

The gene encoding the B7-H3 antibody or antigen-binding fragment thereof of the present invention may be included in an expression vector. The expression vector includes a promoter, a B7-H3 antibody or antigen-binding fragment gene operably linked to the promoter, and a restriction enzyme cleavage site.

The expression vector of the present invention may be a viral vector, a naked DNA or RNA vector, a plasmid, a cosmid or phage vector, a DNA or RNA vector associated with a cationic condensing agent or a DNA or RNA vector encapsulated in the liposome.

Expression vectors of the present invention may be introduced into host cells.

The host cells of the present invention may be animal cells, plant cells, or eukaryotic cells such as eukaryotic microorganisms, and may be, for example, NS0 cells, Vero cells, Hela cells, COS cells, CHO cells, HEK293 cells, BHK cells, MDCKII cells, Sf9 cells and the like.

The host cells of the present invention may be prokaryotic cells, and may be, for example, E. coli or Bacillus subtilis.

The present invention provides a method for preparing a B7-H3 antibody or antigen-binding fragment thereof by culturing the above-described host cells. Culturing may be performed according to methods widely known in the art, and conditions such as a culture temperature, culture time, medium type and pH may be appropriately adjusted depending on the types of the cells.

The method for preparing a B7-H3 antibody or antigen-binding fragment thereof of the present invention may further include separating, purifying, and recovering the produced antibody or antigen-binding fragment thereof. For example, to recover the antibody or antigen-binding fragment thereof, there are available methods such as filtration, affinity chromatography, ion exchange chromatography, hydrophobic chromatography, HPLC and the like.

The present invention provides a pharmaceutical composition for treating or preventing cancer, which includes the above-described B7-H3 antibody or antigen-binding fragment thereof.

The B7-H3 antibody or antigen-binding fragment thereof of the present invention binds to B7-H3 of cancer cells in which B7-H3 is expressed to neutralize (inhibit) the activity of B7-H3, and remove B7-H3 by introducing it into the cells. Thereby, activation of immune cells may be induced, and from this, cancer may be treated.

The cancer of the present invention may be EGFR overexpressing cancer.

The cancer of the present invention may be any one selected from the group consisting of lung cancer (small cell lung cancer and non-small cell lung cancer), breast cancer, ovarian cancer, uterine cancer, cervical cancer, glioma, neuroblastoma, prostate cancer, pancreatic cancer, colorectal cancer, colon cancer, head and neck cancer, leukemia, lymphoma, renal cancer, bladder cancer, gastric cancer, liver cancer, skin cancer, brain tumor, cerebrospinal cancer, adrenal tumor, melanoma, sarcoma (osteosarcoma and soft tissue sarcoma), multiple myeloma, pancreatic neuroendocrine neoplasm, peripheral nerve sheath tumor and small cell tumor.

The pharmaceutical composition of the present invention may be more effective for solid cancer.

The pharmaceutical composition of the present invention may further include a pharmaceutically acceptable carrier, and may be formulated with the carrier. The term “pharmaceutically acceptable carrier” refers to a carrier or diluent that does not stimulate the organism and does not inhibit biological activities and properties of the administered compound.

Pharmaceutically acceptable carriers for liquid compositions include saline, sterile water, Ringer's solution, buffered saline, albumin injectable solutions, dextrose solution, maltodextrin solution, glycerol, ethanol, and a mixture of one or more of these components. If necessary, other conventional additives such as antioxidants, buffers, and bacteriostats may be added to the carrier. In addition, diluents, dispersants, surfactants, binders and lubricants may also be additionally added to formulate the pharmaceutical composition into injectable formulations, pills, capsules, granules or tablets such as aqueous solutions, suspensions, emulsions and the like.

The pharmaceutical composition of the present invention is not limited in the formulation, and may be prepared, for example, in oral or parenteral formulations. More specifically, the formulations include oral, rectal, nasal, topical (including the cheek and sublingual), subcutaneous, vaginal or intramuscular, subcutaneous and intravenous administration. Alternatively, forms suitable for administration by inhalation or insufflations may also be included.

The pharmaceutical composition of the present invention is administered to a subject in a pharmaceutically effective amount. The effective amount may be determined depending on types and severity of disease of the patient, activity of drug, sensitivity to drug, administration time, administration route and rate of release, duration of treatment, factors including concurrent drugs, and other factors well known in the medical field.

The dosage of the pharmaceutical composition of the present invention may vary depending on the weight, age, sex, health conditions or diet of a patient, administration time, administration method, excretion rate and severity of the disease. The appropriate dosage may vary depending on, for example, an amount of drug accumulated in the patient's body and/or the efficacy of the active ingredient of the present invention used.

Generally, the amount may be calculated on the basis of EC₅₀, which is generally determined to be effective in in vivo animal models and in vitro, for example, from 0.01 μg to 1 g per kg of body weight. Further, the pharmaceutical composition of the present invention may be administered once or several times per unit time during unit periods of time such as daily, weekly, monthly or yearly, or may be continuously administered using an infusion pump for a long time. The number of repeated administration doses is determined in consideration of a residential time of drug in the body, a drug concentration in the body, etc. Even after treatment according to the course of disease treatment, the composition may be further administered for preventing recurrence, i.e., relapse of the disease.

The pharmaceutical composition of the present invention may be administered in combination with other anti-cancer substances. For example, the composition may be administered in combination with immuno-anticancer agents such as a PD-1 inhibitor.

The pharmaceutical composition of the present invention may further include a component to maintain or increase the solubility and absorption of the active ingredient. In addition, the pharmaceutical composition may further include chemotherapeutic agents, anti-inflammatory agents, antiviral agents, immunomodulators and the like.

Further, the pharmaceutical composition of the present invention may be formulated using any method known in the art to allow rapid, sustained or delayed release of the active ingredient after administration to a mammal. The formulation may be produced in a form of powders, granules, tablets, emulsions, syrups, aerosols, soft or hard gelatin capsules, sterile injectable solutions, sterile powders.

The present invention provides a method for treating cancer including administering a B7-H3 antibody or antigen-binding fragment thereof, or a gene encoding the same, to a subject. Cancers that can be treated by this method are as described above.

The B7-H3 antibody or antigen-binding fragment thereof of the present invention, or the gene encoding the same, may be administered to a human subject for therapeutic purposes.

The B7-H3 antibody or antigen-binding fragment thereof of the present invention, or the gene encoding the same, may be administered to a non-human mammal expressing B7-H3 for veterinary purposes or as an animal model of human diseases.

The present invention provides the B7-H3 antibody or antigen-binding fragment thereof, which is used as a medicament.

The B7-H3 antibody or antigen-binding fragment thereof of the present invention may be administered to a subject suffering from “a disease or disorder in which B7-H3 activity is detrimental” for therapeutic purposes.

The “disease or disorder in which B7-H3 activity is detrimental” of the present invention includes diseases and disorders in which the presence of B7-H3 in the subject suffering from a specific disease or disorder has been turn out or suspected to be a factor responsible for the pathophysiology of the disorder or contributing to the worsening of the disorder.

The medicament of the present invention may be an anticancer drug for treatment of cancer. The types of cancer are as described above.

Hereinafter, the present invention will be described in more detail through examples.

### Example 1: Binding Force Test of B7-H3 Using ELISA Method

This experiment was conducted to confirm the binding force of the B7-H3 antibody to B7-H3 protein.

Experimental Method

The binding force to B7-H3 was confirmed according to the concentrations of #1 to #9 antibodies by the following method.

After coating a plate with recombinant human B7-H3 protein (Rndsystem, Cat #1027-B3-100) (30 μL, 20 nM) in 1×PBS solution, the plate was covered and coated overnight at 2 to 8° C. Thereafter, the wells were washed once with 150 μL PBS per well, and blocked with 120 μL of blocking buffer (1×PBS-T w/3% BSA) per well for 2 hours at room temperature. The blocking buffer was discarded and 30 μL of antibody solution was added using a series of dilution solutions, and reacted at room temperature for 1 hour, then the wells were washed three times with 150 μL of wash buffer per well. 30 μL of HRP conjugate of anti-HA Tag antibody diluted in the blocking buffer was added to each well, and reacted at room temperature for 1 hour. Thereafter, the wells were washed three times with 150 μL of washing buffer per well, and the HRP reaction was developed using TMB, then the optical density (OD) was measured at 450 nm.

Results

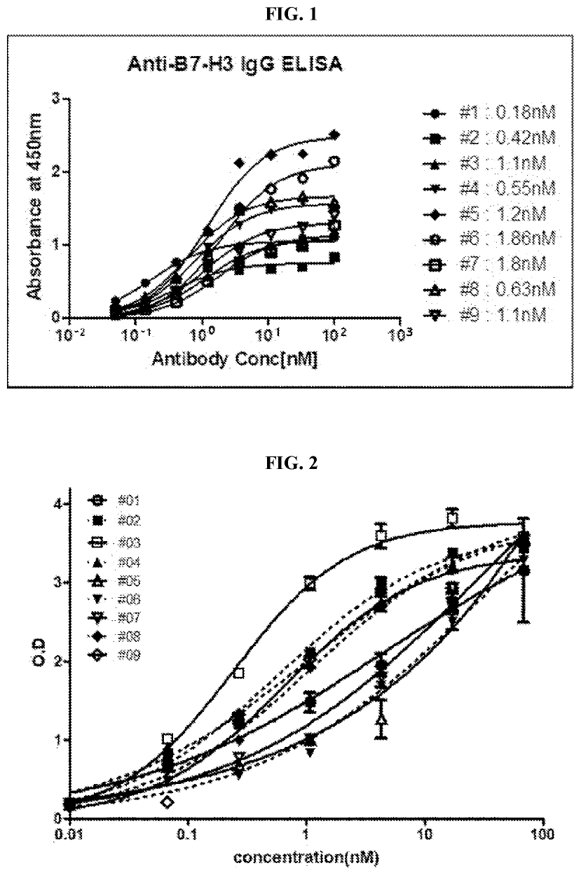

The binding force to B7-H3 according to the concentrations of #1 to #9 antibodies, and the concentrations of the respective antibodies that allow 50% of B7-H3 to exist in an antigen-antibody bound state when #1 to #9 antibodies are treated (EC₅₀) were checked (FIG. 1). It was confirmed that #1 to #9 antibodies specifically bind to B7-H3 with excellent binding force.

### Example 2: Binding Force Test of B7-H3 Antibody Using Cell ELISA Method

This experiment was conducted to confirm the binding force of the B7-H3 antibody to B7-H3 expressed on the cell membrane.

Experimental Method

2.1. Preparation of Reagents

1×PBS and 1×PBS-T (0.05% tween-20) were prepared. A blocking buffer was prepared so that BSA was 3% BSA in 1×PBS-T (0.05% Tween-20). An antibody dilution buffer was prepared so that BSA was 1% BSA in 1×PBS-T (0.05% Tween-20).

2.2. Cell Harvesting and Seeding

After harvesting cells from MCF-7 cell line, RKO cell line, and RKO/B7H3 cell line, the cells were diluted with a culture medium (10% FBS added) so that they could be seeded with 3×10⁴ cells, 100 μL/well to adjust the cell concentration. After seeding at 100 μL/well in a cell culture plate, 96-well plate, followed by culturing overnight in an incubator at 37° C. containing 5% CO₂.

2.3. Cell Fixation and Blocking

1) 100 μL of 8% paraformaldehyde solution was added to the 96-well plate, and centrifuged at 300 g for 10 minutes, then fixed at room temperature for a total of 20 minutes including the centrifugation time.

2) Thereafter, the fixation solution was removed, and the wells were washed by adding 1×PBS at 250 μL/well.

3) After washing, 100 μL/well of blocking buffer was added and incubated at room temperature for 1 hour. After removing the blocking buffer, 1×PBS-T was added at 250 μL/well to wash the wells again, and the PBS-T remaining in the wells was swept off (this washing process was repeated three times).

2.4. Antibody Reaction

After diluting #1 to #9 antibodies by serial dilution method, the diluted samples (antibodies) at the concentrations shown in Table 1 below were dispensed into a 96-well plate in duplicate by 100 μL, so that the antibodies were bound at room temperature for 2 hours. Thereafter, washing was performed.

The concentrations of the antibodies used in this experiment are shown in Table 1 below.

TABLE 1

| Item | #1 to #9 antibodies |
|---|---|
| Concentration | 4 fold serial dilution from |
| (MCF-7 cell ELISA) | 10 μg/mL |
| Concentration | 4 fold serial dilution from |
| (RKO and RKO/B7H3 cell ELISA) | 2.5 μg/mL |

2.5. Detection Antibody Reaction

Peroxidase-AffiniPure Rabbit Anti-Human IgG, F(ab′)2 fragment specific antibody was diluted at a ratio of 1:5,000 using an antibody dilution buffer, and 100 μL was dispensed into each well, and reacted at room temperature for 1 hour. Thereafter, the wells were washed and 100 μL of 1-step TMB substrate solution was dispensed into each well, then reacted at room temperature for 10 minutes by eliminating light. After 10 minutes, 50 μL of 1 N hydrochloric acid was added to each well to stop the TMB reaction, and the OD values were measured at 450 nm.

Results

As a result of confirming the binding affinity of #1 to #9 antibodies to MCF-7 cells according to the concentrations thereof, it was confirmed that #1 to #9 antibodies had excellent binding force to the MCF-7 cell line (FIG. 2 and Table 2).

Table 2 below shows EC₅₀ concentrations of each antibody to MCF-7 cells.

TABLE 2

| Antibodies | EC₅₀ (nM) |
|---|---|
| #1 | 0.605 |
| #2 | 0.061 |
| #3 | 0.197 |
| #4 | 0.502 |
| #5 | 1.740 |
| #6 | 0.234 |
| #7 | 1.012 |
| #8 | 0.159 |
| #9 | 0.322 |

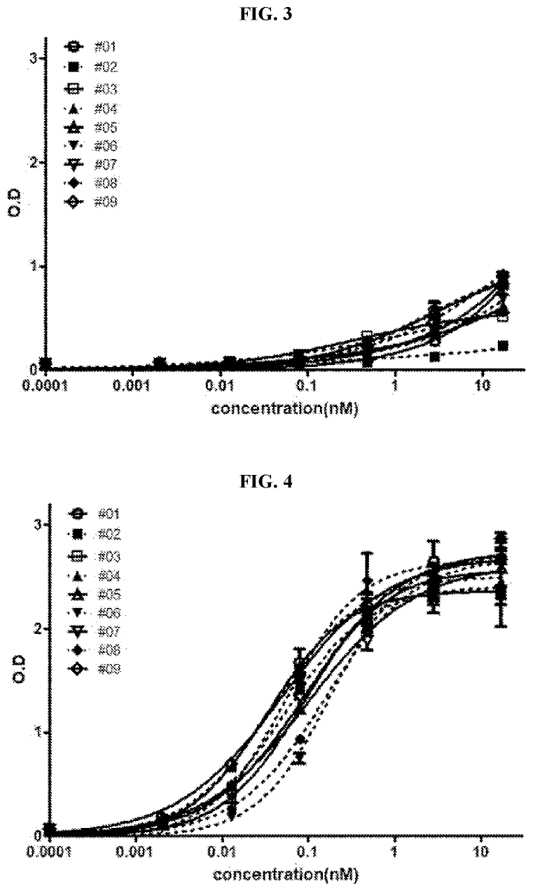

In addition, all #1 to #9 antibodies showed weak binding affinity to the RKO cell line in which B7-H3 was not overexpressed (FIG. 3), but exhibited excellent binding affinity to the RKO/B7H3 cell line in which B7-H3 was overexpressed (FIG. 4 and Table 3).

Table 3 below shows the EC₅₀ concentrations of each antibody to RKO/B7H3 cells.

TABLE 3

| Antibodies | EC₅₀ (nM) |
|---|---|
| #1 | 0.015 |
| #2 | 0.036 |
| #3 | 0.065 |
| #4 | 0.042 |
| #5 | 0.091 |
| #6 | 0.065 |
| #7 | 0.187 |
| #8 | 0.060 |
| #9 | 0.050 |

### Example 3: Cell Internalization Test

This experiment was conducted to confirm B7-H3 internalization ability of the B7-H3 antibody.

Experimental Method

3.1. Antibody-pHAb Amine Reactive Dye Conjugation

After dissolving 0.084 g of sodium bicarbonate in distilled water, the pH was adjusted to 8.5 using a pH meter, and the final volume was adjusted to 100 mL, followed by filtering impurities with a syringe filter, to prepare an amine conjugation buffer (10 mM sodium bicarbonate buffer (pH 8.5)).

The antibody buffer was replaced with the amine conjugation buffer using a desalting column, and a bottom closure of the column was removed, then it was placed in a 1.5 mL microcentrifuge tube (hereinafter referred to as a collection tube). Centrifugation was performed at 1,500 g for 1 minute to remove the storage solution of the column. The collection tube was removed and replaced with a new collection tube.

Thereafter, washing was performed. 300 μL of equilibration buffer (10 mM sodium bicarbonate buffer) was put into the tube, and centrifugation was performed at 1,500 g for 1 minute, then the process was performed twice. After performing the process twice, 300 μL of equilibration buffer (10 mM sodium bicarbonate buffer) was put into the tube, followed by centrifugation at 1,500 g for 2 minutes. Then, 70 μL of each antibody was put and centrifuged at 1,500 g for 2 minutes.

Amine-reactive dye was taken out at −80° C., centrifuged at 14,000 g for 10 seconds to settle down, and DMSO and distilled water were mixed at a ratio of 1:1. Next, 25 μL of the mixture was put into 10 mg/mL, and vortexed for 3 minutes to fully dissolve.

Thereafter, antibody-pHAb amine reactive dye conjugation was performed.

1.2 μL of pHAb amine-reactive dye was put into 100 μg of antibody, then was slowly mixed for 1 hour at room temperature. Then, the antibody and pHAb amine reactive dye conjugation reagent were put into the desalting column, and unreacted dye was removed by centrifugation at 1,500 g for 2 minutes.

The concentrations of the pHAb amine dye and the conjugated antibody were calculated using the following equations.

$$Antibody Concentration ( mg / mL ) = \frac{{A}_{280} - ( {A}_{532} \times 0.256 )}{1.4}$$

$$Dye - to - Antibody Ratio ( DAR ) = \frac{( {A}_{532} \times 150 , 000 )}{Ab Concentration ( \frac{mg}{ml} ) \times 75 , 000}$$

(wherein, molecular weight of antibody=150,000, extinction coefficient of pHAb reactive dye=75,000, and correction factor for pHAb reactive dye=0.256)

3.2. Cell Seeding

After harvesting the MCF-7 cell line, RKO cell line and RKO/B7H3 cell line, the number of cells was counted. The cells were suspended at a cell concentration of 3×10⁵ cells/mL using a culture medium.

The cells were dispensed into a 96-well black, clear-bottom plate by 100 μL so as to be 3×10⁴ cells per well, and incubated for 24 hours in an incubator at 37° C. containing 5% Co₂.

3.3. Conjugation of Primary Antibody to pHAb Amine-Labeled Secondary Antibody

4 μg/mL of primary antibody (control IgG, #1 to #9) and pHAb amine-labeled secondary antibody were put into a RPMI1640 (phenol free, serum free) medium at a ratio of 1:4 and mixed. Then, the mixture was put into a thermostatic water bath at 37° C. and reacted for 1 hour.

3.4. Conjugated Antibody Treatment

After removing the culture medium put when seeding the cells, 100 μL of a solution in which the primary antibody and the pHAb amine-labeled secondary antibody were conjugated was dispensed into each well. Then, a reaction was performed for 24 hours in an incubator at 37° C. containing 5% CO₂.

3.5. Fixation and Washing

8% paraformaldehyde was diluted to 4% paraformaldehyde using 1×PBS. The culture solution treated with the conjugated antibody was removed, and 100 μL of 4% paraformaldehyde was dispensed. The 96-well plate was centrifuged at 300 g for 10 minutes. After centrifugation, a reaction was performed at room temperature for 10 minutes. Then, 250 μL of 1×PBS was added per well and washed three times, then 100 μL of 1×PBS was added per well.

3.6. Analyze

Fluorescence intensities were measured by OD values of Ex 520 nm/Em 565 nm using a microplate reader.

Results

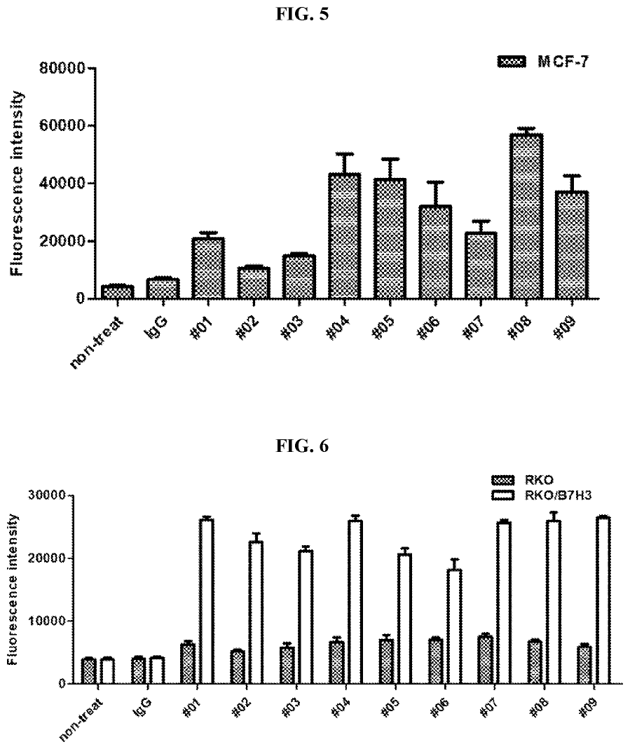

As shown in FIGS. 5 and 6, it was confirmed that #1 to #9 antibodies allowed B7-H3 to be introduced into cells (cell internalization) by binding to the B7-H3 present in the cells (FIGS. 5 and 6).

It was verified that #1 to #9 antibodies had excellent B7-H3 internalization abilities to the MCF-7 cell line, and in particular, it was confirmed that 7 antibodies (#1, #4, #5, #6, #7, #8 and #9) had very excellent B7-H3 internalization ability.

In addition, it was confirmed that the B7-H3 internalization abilities of #1 to #9 antibodies were significantly superior in the RKO/B7H3 cell line in which B7-H3 was overexpressed compared to the RKO cell line in which B7-H3 was not overexpressed.

### Example 4: Invasion Test

This experiment was conducted to confirm the inhibitory effect of the B7-H3 antibody on cancer cell invasion.

Experimental Method

4.1 Preparation of Reagents

Culture medium: It was prepared by putting 50 mL of FBS, 5 mL of antibiotic-antimycotic (100×), 5 mL of NEAA, and 5 mL of sodium pyrubate into 500 mL of RPMI 1640 medium. 1×PBS: It was prepared by mixing 100 mL of 10×PBS in 900 mL of tertiary distilled water. 0.2% crystal violet: After putting 10 mL of 1% crystal violet solution to 40 mL of methanol and mixing by inverting, the mixture was stored at room temperature in a light-blocked state.

4.2. Trasnwell Insertion and Matrigel Coating

Forceps were heated with an alcohol lamp and allowed to cool. Transwell was mounted on an SPL 24-well plate. After dispensing 22 μL of matrigel diluted at a ratio of 1:10 with serum free media (SFM) into each inner well (inside the transwell), allowed to be spread evenly on the membrane. Thereafter, the matrigel was dried at room temperature for 1-2 hours to harden.

4.3. RKO, RKO/B7H3 Seeding and Cultivation

After removing the medium of RKO and RKO/B7H3 cultured in a 10 cm dish, washing with 8 mL of DPBS, 1 mL of trypsin-EDTA (T/E) solution was put, and left in an incubator at 37° C. for 2-3 minutes to remove the cells. The removed cells were collected in a 15 mL tube using 6 mL of SFM, and then centrifuged at 700 rpm for 3 minutes. After removing the supernatant and dissolving the cell pellet with 3 mL of SFM, the number of cells was counted. SFM was added to make the cell concentration be 5×10⁶ cells/mL.

RKO and RKO/B7H3 were slowly put into an insert well by 1×10⁶ cells/200 μL, respectively, and 600 μL of culture medium supplemented with 10% FBS was added to an outer well.

In order to confirm the antibody treatment effect, RKO/B7H3 (1×10⁶ cells/200 μL) cell line was mixed with #1 to #9 antibodies at a concentration of 20 μg/mL, respectively, slowly put into the insert well, and 600 μL of culture medium supplemented with 10% FBS was added to the outer well.

Thereafter, the cells were cultured for 48 hours in an incubator at 37° C. containing 5% CO₂.

4.4. Crystal Violet Staining

600 μL of PBS, 0.2% crystal violet, and tertiary distilled water were dispensed into each well of a 24-well plate.

The cultured cells were taken out and the insert well was turned upside down to remove the medium inside, and then immersed in PBS and washed. Then, the insert well was put into 0.2% crystal violet and stained at room temperature for 30 minutes.

After taking out the insert well and turning it upside down to remove the crystal violet inside, dyeing was stopped by immersing it in tertiary distilled water.

After holding the insert well with forceps and washing it by shaking in the tertiary distilled water contained in a wide bucket, a cotton swab was used to wipe out cells that were not invaded into the inner membrane.

4.5. Photography and Data Analysis

The degree of cell invasion was photographed, and the number of cells invaded was counted using Image J.

Results

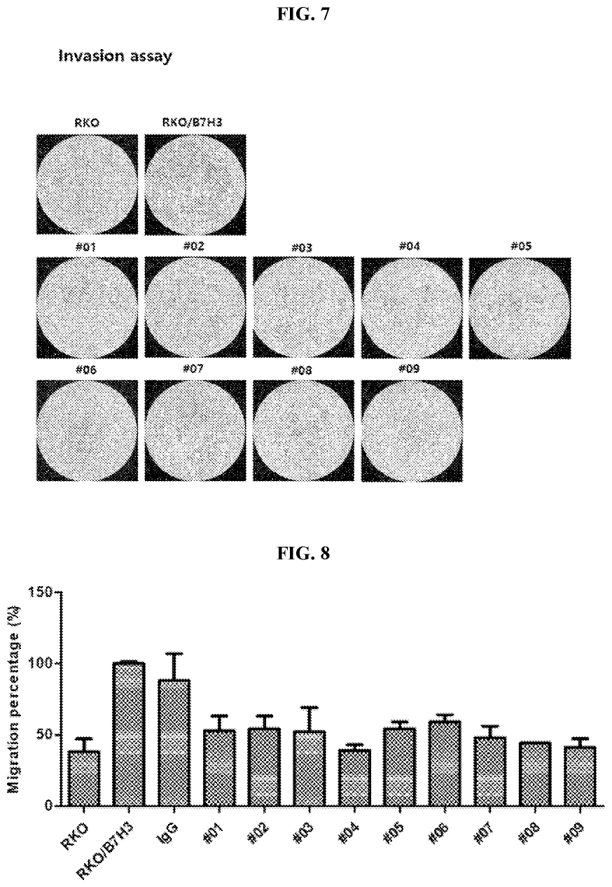

It was confirmed that cancer cell invasion was actively progressed in the RKO/B7H3 cell line compared to the RKO cell line. When the RKO/B7H3 cell line was treated with #1 to #9 antibodies and cultured, it was confirmed that the invasion was reduced by about 60 to 85% (FIGS. 7 and 8). That is, when B7-H3 was overexpressed, it was confirmed that the cancer cell invasion was actively progressed and invasion was inhibited by the B7-H3 antibody, and as a result, it was verified that #1 to #9 antibodies had excellent invasion inhibitory effect.

### Example 5: Migration Test

This experiment was conducted to confirm the inhibitory effect of B7-H3 antibody on cancer cell migration.

Experimental Method

5.1. Preparation of Reagents

Culture medium: It was prepared by putting 50 mL of FBS, 5 mL of antibiotic-antimycotic (100×), 5 mL of NEAA, and 5 mL of sodium pyrubate into 500 mL of RPMI 1640 medium. 1×PBS: It was prepared by mixing 100 mL of 10×PBS in 900 mL of tertiary distilled water. 0.2% crystal violet: After putting 10 mL of 1% crystal violet solution to 40 mL of methanol and mixing by inverting, the mixture was stored at room temperature in a light-blocked state.

5.2. Trasnwell Insertion

Forceps were heated with an alcohol lamp and allowed to cool, and then transwell was mounted on an SPL 24-well plate as much as the amount used.

5.3. RKO, RKO/B7H3 Seeding and Cultivation

After removing the medium of RKO and RKO/B7H3 cultured in a 10 cm dish, and washing with 8 mL of DPBS, 1 mL of T/E solution was put, and left in an incubator at 37° C. for 2-3 minutes to remove the cells. The removed cells were collected in a 15 mL tube using 6 mL of SFM, and then centrifuged at 700 rpm for 3 minutes. After removing the supernatant and dissolving the cell pellet with 3 mL of SFM, the number of cells was counted. SFM was added to make the cell concentration be 1×10⁶ cells/mL, and slowly add 2×10⁵ cells/200 μL cells to an insert well, then add 600 μL of culture medium supplemented with 10% FBS to an outer well. Thereafter, the cells were cultured for 16 hours in an incubator at 37° C. containing 5% CO₂.

5.4. Crystal Violet Staining

600 μL of PBS, 0.2% crystal violet, and tertiary distilled water were dispensed into each well of a 24-well plate.

The cultured cells were taken out and the insert well was turned upside down to remove the medium inside, and then immersed in PBS and washed. Then, the insert well was put into 0.2% crystal violet and stained at room temperature for 30 minutes.

After taking out the insert well and turning it upside down to remove the crystal violet inside, dyeing was stopped by immersing it in tertiary distilled water.

After holding the insert well with forceps and washing it by shaking in the tertiary distilled water contained in a wide bucket, a cotton swab was used to wipe out cells that were not invaded into the inner membrane.

5.5. Photography

The degree of cancer cell migration was confirmed using a microscope, then the cells were photographed.

5.6. Crystal Violet Extraction

After adding 200 μL of 100% methanol to new 24 wells, the insert well that had been photographed was inserted, and 100 μL of 100% methanol was added to inside the insert well, then sealed with parafilm and shaken at room temperature for 1 hour to extract the dyed reagent. The insert well was removed, then 200 μL was scooped out and transferred to a 96-well plate, and the OD values were measured at 590 nm. The degree of migration was compared with the measured OD values.

5.7. Data Analysis

In the case of analyzing the OD values obtained by crystal violet extraction, the OD values of the experimental group was divided based on the value of non-treated RKO/B7H3 cells, and the degree of migration was converted into a percentage value and compared.

Results

It was confirmed that cancer cell migration was actively progressed in the RKO/B7H3 cell line compared to the RKO cell line. Thus, it could be seen that #1 to #9 antibodies had excellent cancer cell migration inhibitory effect on the RKO/B7H3 cell line.

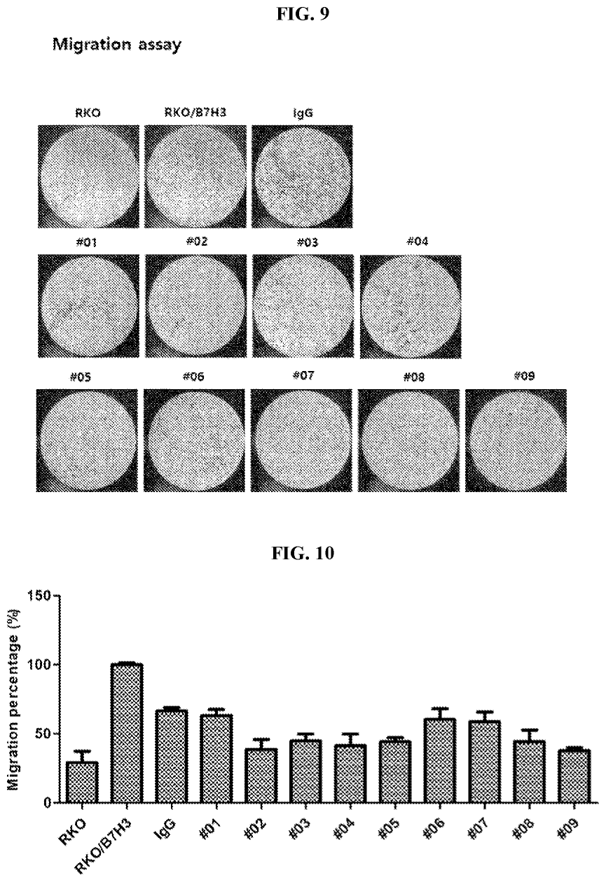

When the cancer cell invasion inhibitory effect and migration inhibitory effect are comprehensively considered, it can be seen that #1 to #9 antibodies have excellent cancer metastasis inhibitory effects (FIGS. 9 and 10).

### Example 6: Epitope Identification Experiment Using Antigen-Binding Fragments (scFv) of #1 to #9 Antibodies

This experiment was conducted to confirm whether the epitopes of #1 to #9 antibodies were identical.

Experimental Method

6.1. Material

3% BSA in PBST as a blocking buffer and 0.05% PBST as a washing buffer were used, and the materials listed in Table 4 below were used.

TABLE 4

| Item | Company | Cat No. | Product name |
|---|---|---|---|
| Coating | Rndsystem | 1027-B3-100 | Recombinant Human |
| Ag. | | | B7-H3 Fc chimera |
| | | | Protein |
| Primary | Self-production of antigen-binding fragments | | |
| antibody | of #1 to #9 (9 types of Anti-B7-H3 scFvs) | | |
| Primary | Self-production of biotinylated antigen-binding fragments | | |
| antibody | of #1 to #9 (9 types of Biotinylated anti-B7-H3 scFvs) | | |
| Secondary | Jackson | 016-030-084 | Peroxidase-conjugated |
| antibody | immunoresearch | | Streptavidin |

6.2. Classification of Antibodies with Identical Epitopes

A 96-well plate was coated with Recombinant B7-H3 protein, and treated with biotinylated scFv having heavy and light chain variable regions of #1 to #9 antibodies to confirm color development. Then, the color development was confirmed when the biotinylated scFv and non-biotinylated scFv were treated together, and by using the principle that the degree of color development is decreased when the binding between the antigen (B7-H3) and the biotinylated scFv is hindered, it was confirmed whether the epitopes of #1 to #9 antibodies were identical.

Results

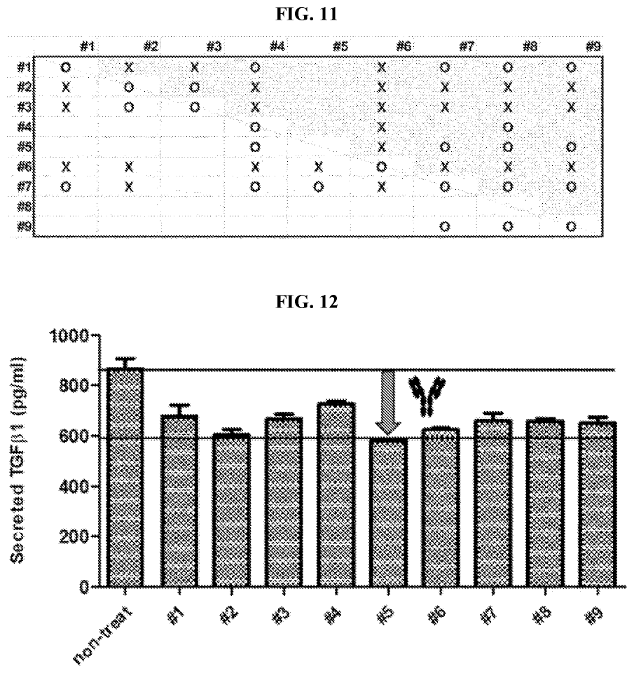

It was confirmed that there was no antibody having the same epitope as #6 antibody, the epitopes of #2 antibody and #3 antibody were identical, and the epitopes of #1, #4, #5, #7, #8 and #9 antibodies were identical (FIG. 11).

### Example 7: Analysis of TGFβ Secretion in B7-H3 Overexpressed Cells

This experiment was conducted to confirm whether the secretion of TGFβ, a representative substance that regulates the tumor microenvironment, was inhibited by the B7-H3 antibody.

Experimental Method

7.1. Preparation of Reagents

1×PBS and washing buffer (1×PBS-T (0.05% tween-20)) were prepared.

A blocking buffer was prepared with BSA so as to be 1% BSA in 1×PBS-T (0.05% Tween-20). The same antibody dilution buffer reagent as the washing buffer was used. A sample neutralization buffer was prepared by adding 25 mL of 1 M HEPES, 12 mL of 5 N NaOH, and 13 mL of tertiary distilled water (autoclaved) based on 50 mL, and stored at 4° C.

7.2. Cell Seeding

After harvesting the RKO/B7H3 cell line, the number of cells was counted. The cells were suspended at a cell concentration of 2×10⁵ cells/mL using a culture medium. The cells were dispensed into a 24-well plate by 500 μL so as to be 1×10⁵ cells per well, and incubated for 24 hours in an incubator at 37° C. containing 5% CO₂.

7.3. Culture Medium Replacement and Supernatant Collection

The cell seeding medium was removed, and SFM was dispensed by 200 μL and removed. 500 μL of SFM was dispensed for each well, and cultured for 24, 48 and 72 hours in an incubator at 37° C. containing 5% CO₂. The supernatants cultured for 24, 48 and 72 hours were collected in a 1.5 mL tube, and centrifuged at 300 g for 3 minutes to settle cell debris. 400 μL of the supernatant was collected in a new 1.5 mL tube and stored at −80° C.

7.4. Capture Antibody Coating

Human TGF-β1 capture antibody (stock concentration: 240 μg/mL, −20° C.) was slowly dissolved on ice in advance, then the human TGF-β1 capture antibody was diluted at a ratio of 1:120 using the coating buffer (PBS) so as to be a concentration of 2 μg/mL. 0.2 μg/well (100 μL/well) was dispensed into each 96-well plate, and then reacted overnight at room temperature.

7.5. Washing and Blocking

After washing three times with 250 μL of washing buffer per well, no solution was left on the plate. Then, the blocking buffer was dispensed at 250 μL/well, and reacted at room temperature for 2 hours.

7.6. Standard Preparation, Sample Activation and Sample Treatment

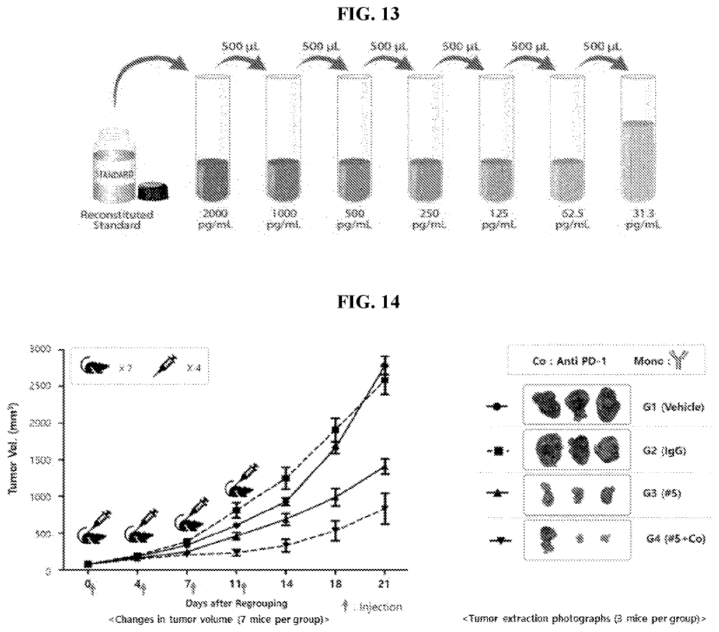

A standard (mouse TGF-β1 (stock concentration: 190 ng/mL, −20° C.) was diluted using an antibody dilution buffer at a ratio of 1:95 so as to be 2,000 μg/mL, followed by performing 2-fold serial dilution (FIG. 13).

After dispensing the sample into a 96-well plate by 100 μL and adding 1 N HCl by 20 μL, the plate was shaken for 10 seconds to mix, and reacted at room temperature for 10 minutes. Thereafter, 1.2 N NaOH/0.5 M HEPES was added by 20 μL to neutralize the mixture. Thereafter, the plate was washed three times with 250 μL of washing buffer per well, and 100 μL of the standard and sample prepared above were dispensed per well, then reacted at room temperature for 2 hours. After the reaction, washing was performed.

7.7. Detection Antibody Reaction

A detection antibody (stock concentration: 3 μg/mL, −20° C.) was diluted using an antibody dilution buffer at a ratio of 1:60 so as to be a concentration of 50 ng/mL. The diluted detection antibody was dispensed by 100 μL/well, and then reacted at room temperature for 2 hours. Thereafter, washing was performed.

7.8. Streptavidin-HRP Reaction

After diluting streptavidin-HRP using the antibody diluent buffer at a ratio of 1:40, the diluted streptavidin-HRP solution was dispensed at 100 μL/well, blocked from light, and reacted at room temperature for 20 minutes. Thereafter, washing was performed.

7.9. Substrate Solution Reaction and OD Value Measurement

100 μL of TMB was dispensed per well, then blocked from light, and reacted at room temperature for 20 minutes. After 20 minutes, the substrate reaction was stopped by adding 50 μL of 1 N HCl as a stop solution to each well. Thereafter, the OD values were measured at 450 nm.

Results

As a result of treating the B7-H3 overexpressed RKO/B7H3 cell line with #1 to #9 antibodies, it was confirmed that the level of secreted TGFβ was inhibited by 25% to 30%, and among them, it was also confirmed that #5 antibody was most excellent.

From this, it can be seen that #1 to #9 antibodies (particularly, #5 antibody) can effectively improve the tumor microenvironment by suppressing the secretion of TGFβ, a representative substance that regulates the tumor microenvironment.

### Example 8: Evaluation of Cancer Model Anticancer Efficacy

### (In Vivo Efficacy Test)

This experiment was conducted to confirm that tumor growth was suppressed in an in vivo mouse cancer model when treated with B7-H3 antibody.

Experimental Method

8.1. Preparation of Animal Model

Female 7-week-old Balb/c mice (Korea Biolink) were used. Cell line CT26-TN cells prepared by overexpressing B7-H3 in CT26 cells, which are mouse colorectal cancer cell lines, were diluted in DPBS at a concentration of 5×10⁶ cells/mL, and 100 μL (5×10⁵ cells) per individual were subcutaneously implanted on the right flank. On the 7th day after cell line implant, the tumor volume was calculated using the following equation by means of an electronic caliper.

Tumor volume (mm³)=<Length (mm)×Width (mm)²>×0.5

8.2. Group Separation

On the 7th day after implanting the tumor cell line, the implanted right tumor was measured, and when the tumor size of most of the subjects reached about 40-120 mm³, sizes of the implanted tumors on both sides of one subject were measured, and group separation was performed according to the Z array method based on the average value of the tumor sizes.

8.3. Antibody Administration

Dose concentration: A #5 antibody administration group −10 mg/kg of #5 antibody; and a #5 antibody and anti-PD-1 antibody combined administration group—10 mg/kg of #5 antibody and anti-PD-1 antibody, respectively.

All test substances were administered intravenously (using an insulin syringe) twice a week, for 2 weeks, a total of 4 times, and negative control substances (vehicle (PBS), and IgG) were also administered in the same way.

TABLE 5

| Item | Company | Cat No. | Product name |
|---|---|---|---|
| Anti-PD-1 | BioXcell | BE016 | InVivoMab Anti-mouse |
| antibody | | | PD-1 (CD279) |

8.4. Measurement of Tumor Size and Weight

After group separation, the sizes of all implanted tumors were measured twice a week for 3 weeks in terms of the tumor volumes. At the same time, the tumor sizes were measured and recorded twice a week after group separation for all animals.

8.5. Autopsy

Based on the day of group separation as day 0, tumors were extracted on day 22, photographs were taken for each individual, and tumor weight was measured.

Results

The growth of the implanted CT26-TN cell line was rapidly increased in the vehicle (PBS) and IgG administration groups as the negative control, but in the #5 antibody administration group, it was confirmed that tumor growth was suppressed from the 7th day after regrouping. In addition, it was confirmed that tumor growth was significantly inhibited in the #5 antibody and anti-PD-1 antibody combined administration group (BioXcell, cat #BE016) (FIG. 14).

### Example 9: Quantification of TGFβ in Serum in a Mouse Cancer Model

This experiment was conducted to confirm TFGβ changes in serum in an in vivo mouse cancer model after B7-H3 antibody treatment.

Experimental Method

9.1. Material

Mouse TGF-beta1 DuoSet ELISA kit (cat #: DY1679)

Serum isolated from mice that had completed the in vivo cancer model anticancer test

9.2. Preparation of Antibody

Mouse TGFβ1 capture antibody was diluted in PBS at 1/120, mouse TGFβ1 detection antibody was diluted in PBS at 1/60, and streptavidin-HRP was diluted in PBS at 1/40.

9.3. Preparation of Serum Sample

10 μL of 1 N HCl was added to 40 μL of serum, then shaken at RT for 10 seconds and incubated for 10 minutes. Thereafter, 10 μL of 1.2 N NaOH/0.5 M HEPES was added to stop the reaction, and diluted with PBS at 1/2.

9.4. ELISA Assay

One day before (15-18 hours ago) the assay, the diluted capture antibody was dispensed into a 96-well plate by 100 μL, incubated at room temperature, and washed with 200 μL of PBST (PBS+0.05% Tween 20).

150 μL of blocking solution (PBS+5% Tween 20) was put and incubated for 1 hour at room temperature, followed by washing with 200 μL of PBST. Thereafter, the standard solution provided in the kit and serum sample prepared in advance were dispensed into a 96-well plate by 100 μL in duplication, and reacted at room temperature for 2 hours, followed by washing with 200 μL of PBST.

100 μL of detection antibody was dispensed into wells, and reacted at room temperature for 2 hours, followed by washing with 200 μL of PBST.

100 μL of streptavidin-HRP was dispensed into wells, and reacted at room temperature for 20 minutes, followed by washing with 200 μL of PBST.

After dispensing 100 μL of TMB solution, the color development reaction was performed at room temperature in a state in which light was blocked. Then, 50 μL of 1 N HCl was put to stop the color development reaction. Finally, the OD values were measured at 450 nm.

Results

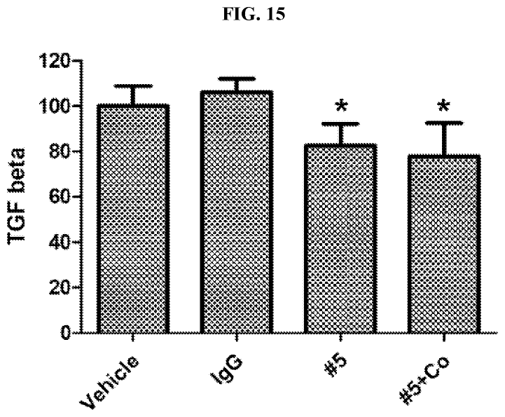

It was confirmed that the concentrations of TGFβ in mouse serum in the #5 antibody administration group and the #5 antibody and anti-PD-1 antibody combined administration group were decreased significantly (based on the vehicle group, p value <0.5) compared to the vehicle (PBS) and IgG administration groups as the negative control (FIG. 15).

### Example 10: Tumor Infiltrating Lymphocytes (TIL) Assay

This experiment was conducted to confirm whether infiltration ability of lymphocyte, an immune cell in cancer tissues, was increased in an in vivo mouse cancer model when treated with B7-H3 antibody.

Experimental Method

10.1. Tumor Cell Isolation

10 mL of DPBS was added to tumors extracted from mice that had completed the in vivo cancer model anticancer test and washed, then the remaining blood was removed.

6 mL of RPMI-1640 (Hyclone, cat. CM058-050) medium was put, and cut finely with scissors, then 600 μL of digestion solution (50 mL RPMI-1640+100 mg collagenase D (Merck, cat. 11088858001)+10 mg DNase I (Sigma-Aldrich, cat. D4513) ) was added, followed by reaction at 37° C. and 120 rpm for 1 hour.

After putting it into a 70 μm of a cell strainer (SPL, cat. SPL93070) and filtering large tissues, 1 mL thereof was placed in a 15 mL tube and centrifuged at 15° C. and 2,000 rpm for 10 minutes to remove the supernatant. After washing once with DPBS, 500 μL of 1×RBC lysis buffer (BioLegend, cat. 420301) diluted in distilled water was added to release the pellet, and the mixture was reacted at room temperature for 5 minutes.

After washing twice in DPBS by the same method as above, the pellet was well dissolved in FACS buffer (DPBS+1% FBS+0.1% sodium azide) to prepare cells.

10.2. FACS Analysis Antibody Information

BioLegend products was used as antibodies for FACS analysis, and information thereof is shown in Table 6 below.

TABLE 6

| Analysis item | Antibody used | Cat No. |
|---|---|---|
| CD4 | APC anti-mouse CD4 | 116014 |
| CD8 | APC/Cyanine7 anti-mouse | 140422 |
| | CD8b.2 | |
| CD3 | FITC anti-mouse CD3 | 100204 |
| CD45 | PE anti-mouse CD45 | 103106 |

10.3. Fluorescence Staining

For single cells of the tumor isolated according to the cell separation test method, purified rat anti-mouse CD16/CD32 (Mouse BD Fc Block™, cat. 553141, BD biosciences) was pretreated for 10 minutes to perform FC blocking, and then the antibodies diluted in FACS buffer (DPBS+1% FBS+0.1% sodium azide) at a dilution factor indicated in the data sheet provided, followed by reaction at 4° C. for 1 hour while blocking light.

The cells after completion of the reaction were washed twice using FACS buffer and then fixed using 2% paraformaldehyde (PFA). The stained cells were measured using a flow cytometer (Atune, Thermo Fisher Scientific) and analyzed using FLOWJO™ V10 (Flowjo, LLC).

Results

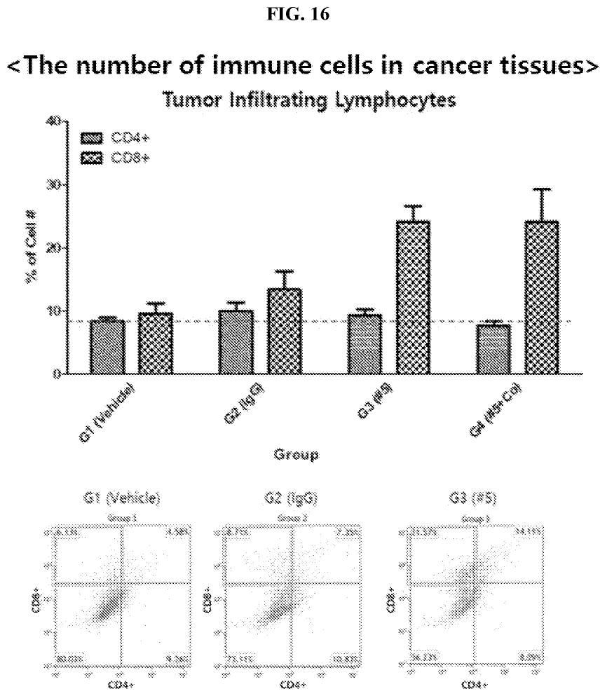

As a result of FACS analysis on CD4+ and CD8+ T cells of tumors extracted from mice, it was confirmed that the infiltration abilities of CD8+ TIL immune cells into cancer tissues were increased in the #5 antibody administration group and the #5 antibody and anti-PD-1 antibody combined administration group compared to the vehicle (PBS) and IgG administration groups. In contrast, it was confirmed that there was no difference in CD4+ T cells between the negative control and the antibody administration groups. From this, it can be seen that cytotoxic lymphocytes (CD8+ T cells) can infiltrate into the cancer tissues to exhibit cytotoxic effects on the cancer cells (see FIG. 16).

Reference to an Electronic Sequence Listing

A sequence listing electronically submitted on Sep. 26, 2023, as a XML file name 20230926 LC0652317 TU SEQ. TXT, created on Sep. 22, 2023 and having a size of 266,319 bytes, is incorporated herein by reference in its entirety.

1: A B7-H3 antibody or antigen-binding fragment thereof comprising a heavy chain variable region which comprises heavy chain complementarity determining regions (HCDRs) below and a light chain variable region which comprises light chain complementarity determining regions (LCDRs) below:
(a) HCDRs of SEQ ID NOs: 1, 10 and 19 and LCDRs of SEQ ID NOs: 28, 37 and 45;
(b) HCDRs of SEQ ID NOs: 2, 11 and 20 and LCDRs of SEQ ID NOs: 29, 38 and 46;
(c) HCDRs of SEQ ID NOs: 3, 12 and 21 and LCDRs of SEQ ID NOs: 30, 39 and 47;
(d) HCDRs of SEQ ID NOs: 4, 13 and 22 and LCDRs of SEQ ID NOs: 31, 40 and 48;
(e) HCDRs of SEQ ID NOs: 5, 14 and 23 and LCDRs of SEQ ID NOs: 32, 41 and 49;
(f) HCDRs of SEQ ID NOs: 6, 15 and 24 and LCDRs of SEQ ID NOs: 33, 42 and 50;
(g) HCDRs of SEQ ID NOs: 7, 16 and 25 and LCDRs of SEQ ID NOs: 34, 43 and 51;
(h) HCDRs of SEQ ID NOs: 8, 17 and 26 and LCDRs of SEQ ID NOs: 35, 44 and 52; or
(i) HCDRs of SEQ ID NOs: 9, 18 and 27 and LCDRs of SEQ ID NOs: 36, 42 and 53.

2: The B7-H3 antibody or antigen-binding fragment thereof according to claim 1, wherein the heavy chain variable region comprises any one framework sequence selected from the group consisting of heavy chain framework sequences (HFRs) below:
(hf1) HFRs of SEQ ID NOs: 54, 63, 68 and 334;
(hf2) HFRs of SEQ ID NOs: 55, 63, 69 and 334;
(hf3) HFRs of SEQ ID NOs: 56, 64, 70 and 334;
(hf4) HFRs of SEQ ID NOs: 56, 64, 71 and 334;
(hf5) HFRs of SEQ ID NOs: 57, 64, 70 and 334;
(hf6) HFRs of SEQ ID NOs: 58, 64, 72 and 334;
(hf7) HFRs of SEQ ID NOs: 59, 65, 73 and 334;
(hf8) HFRs of SEQ ID NOs: 60, 65, 73 and 334;
(hf9) HFRs of SEQ ID NOs: 61, 66, 74 and 334; and
(hf10) HFRs of SEQ ID NOs: 62, 67, 75 and 334.

3: The B7-H3 antibody or antigen-binding fragment thereof according to claim 1, wherein the light chain variable region comprises any one framework sequence selected from the group consisting of light chain framework sequences (LFRs) below:
(lf1) LFRs of SEQ ID NOs: 76, 82, 86 and 335;
(lf2) LFRs of SEQ ID NOs: 77, 82, 87 and 335;
(lf3) LFRs of SEQ ID NOs: 78, 83, 88 and 335;
(lf4) LFRs of SEQ ID NOs: 79, 84, 89 and 335;
(lf5) LFRs of SEQ ID NOs: 80, 84, 90 and 335;
(lf6) LFRs of SEQ ID NOs: 80, 84, 91 and 335;
(lf7) LFRs of SEQ ID NOs: 81, 85, 92 and 335;
(lf0) LFRs of SEQ ID NOs: 93, 98, 101 and 336;
(lf9) LFRs of SEQ ID NOs: 93, 98, 102 and 336;
(lf10) LFRs of SEQ ID NOs: 93, 98, 103 and 336;
(lf11) LFRs of SEQ ID NOs: 93, 98, 104 and 336;
(lf12) LFRs of SEQ ID NOs: 94, 98, 105 and 336;
(lf13) LFRs of SEQ ID NOs: 95, 99, 106 and 336;
(lf14) LFRs of SEQ ID NOs: 96, 99, 107 and 336; and
(lf15) LFRs of SEQ ID NOs: 97, 100, 108 and 336.

4: The B7-H3 antibody or antigen-binding fragment thereof according to claim 1, wherein the heavy chain variable region is any one selected from the group consisting of SEQ ID NOs: 127, 128, 129, 130, 131, 132, 135, 142 and 152.

5: The B7-H3 antibody or antigen-binding fragment thereof according to claim 1, wherein the light chain variable region is any one selected from the group consisting of SEQ ID NOs: 211, 221, 223, 224, 225, 231, 307, 309 and 317.

6: A gene encoding the B7-H3 antibody or antigen-binding fragment thereof of claim 1.

7: A cell comprising a vector introduced therein, in which the gene of claim 6 is inserted.

8: A method for preparing a B7-H3 antibody or antigen-binding fragment thereof comprising culturing the cell of claim 7.

9: A pharmaceutical composition for treating or preventing cancer comprising the B7-H3 antibody or antigen-binding fragment thereof of claim 1.

10: The pharmaceutical composition according to claim 9, wherein the cancer is any one selected from the group consisting of lung cancer, breast cancer, ovarian cancer, uterine cancer, cervical cancer, glioma, neuroblastoma, prostate cancer, pancreatic cancer, colorectal cancer, colon cancer, head and neck cancer, leukemia, lymphoma, renal cancer, bladder cancer, gastric cancer, liver cancer, skin cancer, brain tumor, cerebrospinal cancer, adrenal tumor, melanoma, sarcoma, multiple myeloma, pancreatic neuroendocrine neoplasm, peripheral nerve sheath tumor and small cell tumor.

11: A method for treating cancer, the method comprising administering the B7-H3 antibody or antigen-binding fragment thereof of claim 1, or a gene encoding the same, to a subject.

12: The method according to claim 11, wherein the cancer is any one selected from the group consisting of lung cancer, breast cancer, ovarian cancer, uterine cancer, cervical cancer, glioma, neuroblastoma, prostate cancer, pancreatic cancer, colorectal cancer, colon cancer, head and neck cancer, leukemia, lymphoma, renal cancer, bladder cancer, gastric cancer, liver cancer, skin cancer, brain tumor, cerebrospinal cancer, adrenal tumor, melanoma, sarcoma, multiple myeloma, pancreatic neuroendocrine neoplasm, peripheral nerve sheath tumor and small cell tumor.

13: The B7-H3 antibody or antigen-binding fragment thereof of claim 1, wherein B7-H3 antibody or antigen-binding fragment thereof is used as a medicament for treatment of cancer.

14: The B7-H3 antibody or antigen-binding fragment thereof according to claim 13, wherein the medicament is an anticancer drug.

15: The B7-H3 antibody or antigen-binding fragment thereof according to claim 13, wherein the cancer is any one selected from the group consisting of lung cancer, breast cancer, ovarian cancer, uterine cancer, cervical cancer, glioma, neuroblastoma, prostate cancer, pancreatic cancer, colorectal cancer, colon cancer, head and neck cancer, leukemia, lymphoma, renal cancer, bladder cancer, gastric cancer, liver cancer, skin cancer, brain tumor, cerebrospinal cancer, adrenal tumor, melanoma, sarcoma, multiple myeloma, pancreatic neuroendocrine neoplasm, peripheral nerve sheath tumor and small cell tumor.

16: The method of claim 11, wherein the heavy chain variable region comprises any one framework sequence selected from the group consisting of heavy chain framework sequences (HFRs) below:
(hf1) HFRs of SEQ ID NOs: 54, 63, 68 and 334;
(hf2) HFRs of SEQ ID NOs: 55, 63, 69 and 334;
(hf3) HFRs of SEQ ID NOs: 56, 64, 70 and 334;
(hf4) HFRs of SEQ ID NOs: 56, 64, 71 and 334;
(hf5) HFRs of SEQ ID NOs: 57, 64, 70 and 334;
(hf6) HFRs of SEQ ID NOs: 58, 64, 72 and 334;
(hf7) HFRs of SEQ ID NOs: 59, 65, 73 and 334;
(hf8) HFRs of SEQ ID NOs: 60, 65, 73 and 334;
(hf9) HFRs of SEQ ID NOs: 61, 66, 74 and 334; and
(hf10) HFRs of SEQ ID NOs: 62, 67, 75 and 334.

17: The method of claim 11, wherein the light chain variable region comprises any one framework sequence selected from the group consisting of light chain framework sequences (LFRs) below:
(lf1) LFRs of SEQ ID NOs: 76, 82, 86 and 335;
(lf2) LFRs of SEQ ID NOs: 77, 82, 87 and 335;
(lf3) LFRs of SEQ ID NOs: 78, 83, 88 and 335;
(lf4) LFRs of SEQ ID NOs: 79, 84, 89 and 335;
(lf5) LFRs of SEQ ID NOs: 80, 84, 90 and 335;
(lf6) LFRs of SEQ ID NOs: 80, 84, 91 and 335;
(lf7) LFRs of SEQ ID NOs: 81, 85, 92 and 335;
(lf0) LFRs of SEQ ID NOs: 93, 98, 101 and 336;
(lf9) LFRs of SEQ ID NOs: 93, 98, 102 and 336;
(if10) LFRs of SEQ ID NOs: 93, 98, 103 and 336;
(lf11) LFRs of SEQ ID NOs: 93, 98, 104 and 336;
(lf12) LFRs of SEQ ID NOs: 94, 98, 105 and 336;
(lf13) LFRs of SEQ ID NOs: 95, 99, 106 and 336;
(lf14) LFRs of SEQ ID NOs: 96, 99, 107 and 336; and
(if15) LFRs of SEQ ID NOs: 97, 100, 108 and 336.

18: The method of claim 11, wherein the heavy chain variable region is any one selected from the group consisting of SEQ ID NOs: 127, 128, 129, 130, 131, 132, 135, 142 and 152.

19: The method of claim 11, wherein the light chain variable region is any one selected from the group consisting of SEQ ID NOs: 211, 221, 223, 224, 225, 231, 307, 309 and 317.


SEQUENCE LISTING (336 sequences)

SEQ ID NO: 1 - protein, 5 aa, Artificial Sequence
  HCDR1
SYAMS

SEQ ID NO: 2 - protein, 5 aa, Artificial Sequence
  HCDR1
AYYMI

SEQ ID NO: 3 - protein, 5 aa, Artificial Sequence
  HCDR1
TYYMT

SEQ ID NO: 4 - protein, 5 aa, Artificial Sequence
  HCDR1
SYGMH

SEQ ID NO: 5 - protein, 5 aa, Artificial Sequence
  HCDR1
TYAMS

SEQ ID NO: 6 - protein, 5 aa, Artificial Sequence
  HCDR1
TYAIS

SEQ ID NO: 7 - protein, 5 aa, Artificial Sequence
  HCDR1
NFWIG

SEQ ID NO: 8 - protein, 5 aa, Artificial Sequence
  HCDR1
SYWIS

SEQ ID NO: 9 - protein, 5 aa, Artificial Sequence
  HCDR1
SYAMN

SEQ ID NO: 10 - protein, 17 aa, Artificial Sequence
  HCDR2
TITGSGGSTYYADSVKG

SEQ ID NO: 11 - protein, 17 aa, Artificial Sequence
  HCDR2
ITNPSGGVTSYAQKFQG

SEQ ID NO: 12 - protein, 17 aa, Artificial Sequence
  HCDR2
IINPRGGSTSYAEKFQG

SEQ ID NO: 13 - protein, 17 aa, Artificial Sequence
  HCDR2
AISGSGTSIYYANSVKS

SEQ ID NO: 14 - protein, 17 aa, Artificial Sequence
  HCDR2
AISGSGGSTLYADSVKG

SEQ ID NO: 15 - protein, 16 aa, Artificial Sequence
  HCDR2
SIYYSGYTHYNPSLES

SEQ ID NO: 16 - protein, 17 aa, Artificial Sequence
  HCDR2
WINPPGSITSYADSVKG

SEQ ID NO: 17 - protein, 16 aa, Artificial Sequence
  HCDR2
EISPSGNTNYNPSLKS

SEQ ID NO: 18 - protein, 17 aa, Artificial Sequence
  HCDR2
TISGSGGQTYYADSVKG

SEQ ID NO: 19 - protein, 10 aa, Artificial Sequence
  HCDR3
ARYKSGAFDI

SEQ ID NO: 20 - protein, 10 aa, Artificial Sequence
  HCDR3
ARVGGRGFDI

SEQ ID NO: 21 - protein, 9 aa, Artificial Sequence
  HCDR3
ARWNLGMDV

SEQ ID NO: 22 - protein, 9 aa, Artificial Sequence
  HCDR3
ARGAGWFDY

SEQ ID NO: 23 - protein, 12 aa, Artificial Sequence
  HCDR3
ARGGSATWAFDL

SEQ ID NO: 24 - protein, 10 aa, Artificial Sequence
  HCDR3
ARGYSGWMDV

SEQ ID NO: 25 - protein, 10 aa, Artificial Sequence
  HCDR3
AREVGDVLDY

SEQ ID NO: 26 - protein, 10 aa, Artificial Sequence
  HCDR3
AREVGDLLDY

SEQ ID NO: 27 - protein, 11 aa, Artificial Sequence
  HCDR3
ARWRSIWAFDY

SEQ ID NO: 28 - protein, 11 aa, Artificial Sequence
  LCDR1
RASQGIGNYLG

SEQ ID NO: 29 - protein, 11 aa, Artificial Sequence
  LCDR1
HASQGISNYVA

SEQ ID NO: 30 - protein, 11 aa, Artificial Sequence
  LCDR1
RVSQSISSYLN

SEQ ID NO: 31 - protein, 13 aa, Artificial Sequence
  LCDR1
SGSTSNIGSNTVN

SEQ ID NO: 32 - protein, 13 aa, Artificial Sequence
  LCDR1
SGSSSNIGSNYVY

SEQ ID NO: 33 - protein, 11 aa, Artificial Sequence
  LCDR1
GGNNIGSKSVH

SEQ ID NO: 34 - protein, 14 aa, Artificial Sequence
  LCDR1
TGTSSDIGRYNYVS

SEQ ID NO: 35 - protein, 11 aa, Artificial Sequence
  LCDR1
SGSNIGNNYVS

SEQ ID NO: 36 - protein, 11 aa, Artificial Sequence
  LCDR1
SDNNIGSKSVH

SEQ ID NO: 37 - protein, 7 aa, Artificial Sequence
  LCDR2
TASSLQS

SEQ ID NO: 38 - protein, 7 aa, Artificial Sequence
  LCDR2
DASNRAT

SEQ ID NO: 39 - protein, 7 aa, Artificial Sequence
  LCDR2
DASHRAT

SEQ ID NO: 40 - protein, 7 aa, Artificial Sequence
  LCDR2
GKNIRPN

SEQ ID NO: 41 - protein, 7 aa, Artificial Sequence
  LCDR2
RNNQRPS

SEQ ID NO: 42 - protein, 7 aa, Artificial Sequence
  LCDR2
DNNKRPS

SEQ ID NO: 43 - protein, 7 aa, Artificial Sequence
  LCDR2
RNTQRPS

SEQ ID NO: 44 - protein, 7 aa, Artificial Sequence
  LCDR2
QDSKRPS

SEQ ID NO: 45 - protein, 10 aa, Artificial Sequence
  LCDR3
QQGYSTPSWT

SEQ ID NO: 46 - protein, 9 aa, Artificial Sequence
  LCDR3
QQSYSTLLT

SEQ ID NO: 47 - protein, 9 aa, Artificial Sequence
  LCDR3
QQYYAPSVT

SEQ ID NO: 48 - protein, 11 aa, Artificial Sequence
  LCDR3
QSYDSSLSGWV

SEQ ID NO: 49 - protein, 11 aa, Artificial Sequence
  LCDR3
GTWDSSLSGVV

SEQ ID NO: 50 - protein, 11 aa, Artificial Sequence
  LCDR3
AAWDDSLNVPV

SEQ ID NO: 51 - protein, 11 aa, Artificial Sequence
  LCDR3
GTWDSSLNAYV

SEQ ID NO: 52 - protein, 11 aa, Artificial Sequence
  LCDR3
LVWDRSSDHFV

SEQ ID NO: 53 - protein, 11 aa, Artificial Sequence
  LCDR3
GTWDDSLSGPV

SEQ ID NO: 54 - protein, 30 aa, Artificial Sequence
  VH_FR1
QVQLVQSGAEVKKPGSSVKVSCKASGGTFS

SEQ ID NO: 55 - protein, 30 aa, Artificial Sequence
  VH_FR1
QVQLVQSGAEVKKPGASVKVSCKASGYTFT

SEQ ID NO: 56 - protein, 30 aa, Artificial Sequence
  VH_FR1
EVQLLESGGGLVQPGGSLRLSCAASGFTFS

SEQ ID NO: 57 - protein, 30 aa, Artificial Sequence
  VH_FR1
EVQLLESGGGLVQPGSSLRLSCAASGFTFS

SEQ ID NO: 58 - protein, 30 aa, Artificial Sequence
  VH_FR1
EVQLVESGGGLVQPGGSLRLSCAASGFTFS

SEQ ID NO: 59 - protein, 30 aa, Artificial Sequence
  VH_FR1
QVQLQQWGAGLLKPSETLSLTCAVYGGSFS

SEQ ID NO: 60 - protein, 30 aa, Artificial Sequence
  VH_FR1
QVQLQESGPGLVKPSETLSLTCTVSGGSIS

SEQ ID NO: 61 - protein, 30 aa, Artificial Sequence
  VH_FR1
EVQLVQSGAEVKKPGESLKISCKGSGYSFT

SEQ ID NO: 62 - protein, 30 aa, Artificial Sequence
  VH_FR1
QVQLQQSGPGLVKPSQTLSLTCAISGDSVS

SEQ ID NO: 63 - protein, 14 aa, Artificial Sequence
  VH_FR2
WVRQAPGQGLEWMG

SEQ ID NO: 64 - protein, 14 aa, Artificial Sequence
  VH_FR2
WVRQAPGKGLEWVS

SEQ ID NO: 65 - protein, 14 aa, Artificial Sequence
  VH_FR2
WIRQPPGKGLEWIG

SEQ ID NO: 66 - protein, 14 aa, Artificial Sequence
  VH_FR2
WVRQMPGKGLEWMG

SEQ ID NO: 67 - protein, 16 aa, Artificial Sequence
  VH_FR2
WNWIRQSPSRGLEWLG

SEQ ID NO: 68 - protein, 30 aa, Artificial Sequence
  VH_FR3
RVTITADESTSTAYMELSSLRSEDTAVYYC

SEQ ID NO: 69 - protein, 30 aa, Artificial Sequence
  VH_FR3
RVTMTTDTSTSTAYMELRSLRSDDTAVYYC

SEQ ID NO: 70 - protein, 30 aa, Artificial Sequence
  VH_FR3
RFTISRDNSKNTLYLQMNSLRAEDTAVYYC

SEQ ID NO: 71 - protein, 30 aa, Artificial Sequence
  VH_FR3
RFTISRDNSKDTLYLQMNSLRAEDTAVYYC

SEQ ID NO: 72 - protein, 30 aa, Artificial Sequence
  VH_FR3
RFTISRDNAKNSLYLQMNSLRDEDTAVYYC

SEQ ID NO: 73 - protein, 30 aa, Artificial Sequence
  VH_FR3
RVTISVDTSKNQFSLKLSSVTAADTAVYYC

SEQ ID NO: 74 - protein, 30 aa, Artificial Sequence
  VH_FR3
QVTISADKSISTAYLQWSSLKASDTAMYYC

SEQ ID NO: 75 - protein, 30 aa, Artificial Sequence
  VH_FR3
RITINPDTSKNQFSLQLNSVTPEDTAVYYC

SEQ ID NO: 76 - protein, 23 aa, Artificial Sequence
  VL(kappa)_FR1
DIQMTQSPSSLSASVGDRVTITC

SEQ ID NO: 77 - protein, 23 aa, Artificial Sequence
  VL(kappa)_FR1
DIQMTQSPSFLSASVGDRVTITC

SEQ ID NO: 78 - protein, 23 aa, Artificial Sequence
  VL(kappa)_FR1
DIVMTQSPLSLPVTPGEPASISC

SEQ ID NO: 79 - protein, 23 aa, Artificial Sequence
  VL(kappa)_FR1
EIVMTQSPATLSVSPGERATLSC

SEQ ID NO: 80 - protein, 23 aa, Artificial Sequence
  VL(kappa)_FR1
EIVLTQSPGTLSLSPGERATLSC

SEQ ID NO: 81 - protein, 23 aa, Artificial Sequence
  VL(kappa)_FR1
DIVMTQSPDSLAVSLGERATINC

SEQ ID NO: 82 - protein, 15 aa, Artificial Sequence
  VL(kappa)_FR2
WYQQKPGKAPKLLIY

SEQ ID NO: 83 - protein, 15 aa, Artificial Sequence
  VL(kappa)_FR2
WYLQKPGQSPQLLIY

SEQ ID NO: 84 - protein, 15 aa, Artificial Sequence
  VL(kappa)_FR2
WYQQKPGQAPRLLIY

SEQ ID NO: 85 - protein, 15 aa, Artificial Sequence
  VL(kappa)_FR2
WYQQKPGQPPKLLIY

SEQ ID NO: 86 - protein, 32 aa, Artificial Sequence
  VL(kappa)_FR3
GVPSRFSGSGSGTDFTFTISSLQPEDIATYYC

SEQ ID NO: 87 - protein, 32 aa, Artificial Sequence
  VL(kappa)_FR3
GVPSRFSGSGSGTDFTLTISSLQPEDFATYYC

SEQ ID NO: 88 - protein, 32 aa, Artificial Sequence
  VL(kappa)_FR3
GVPDRFSGSGSGTDFTLKISRVEAEDVGVYYC

SEQ ID NO: 89 - protein, 32 aa, Artificial Sequence
  VL(kappa)_FR3
GIPARFSGSGSGTEFTLTISSLQSEDFAVYYC

SEQ ID NO: 90 - protein, 32 aa, Artificial Sequence
  VL(kappa)_FR3
GIPDRFSGSGSGTDFTLTISRLEPEDFAVYYC

SEQ ID NO: 91 - protein, 32 aa, Artificial Sequence
  VL(kappa)_FR3
GIPDRFSGSGSGTDFTLTISHLEPEDFAVYYC

SEQ ID NO: 92 - protein, 32 aa, Artificial Sequence
  VL(kappa)_FR3
GVPDRFSGSGSGTDFTLTISSLQAEDVAVYYC

SEQ ID NO: 93 - protein, 22 aa, Artificial Sequence
  VL(lambda)_FR1
QSVLTQPPSASGTPGQRVTISC

SEQ ID NO: 94 - protein, 22 aa, Artificial Sequence
  VL(lambda)_FR1
QSVLTQPPSVSAAPGQKVTISC

SEQ ID NO: 95 - protein, 22 aa, Artificial Sequence
  VL(lambda)_FR1
QSALTQPASVSGSPGQSITISC

SEQ ID NO: 96 - protein, 22 aa, Artificial Sequence
  VL(lambda)_FR1
QSALTQPRSVSGSPGQSVTISC

SEQ ID NO: 97 - protein, 22 aa, Artificial Sequence
  VL(lambda)_FR1
SYELTQPPSVSVSPGQTASITC

SEQ ID NO: 98 - protein, 15 aa, Artificial Sequence
  VL(lambda)_FR2
WYQQLPGTAPKLLIY

SEQ ID NO: 99 - protein, 15 aa, Artificial Sequence
  VL(lambda)_FR2
WYQQHPGKAPKLMIY

SEQ ID NO: 100 - protein, 15 aa, Artificial Sequence
  VL(lambda)_FR2
WYQQKPGQSPVLVIY

SEQ ID NO: 101 - protein, 32 aa, Artificial Sequence
  VL(lambda)_FR3
GVPDRFSGSKSGTSASLAISGLQSEDEADYYC

SEQ ID NO: 102 - protein, 32 aa, Artificial Sequence
  VL(lambda)_FR3
GVPDRFSGSKSGTSASLAISGLHSEDEADYYC

SEQ ID NO: 103 - protein, 32 aa, Artificial Sequence
  VL(lambda)_FR3
GVPDRFSGSKSGTSASLAISGLRSEDEADYYC

SEQ ID NO: 104 - protein, 32 aa, Artificial Sequence
  VL(lambda)_FR3
GVPNRFSGSKSGTSASLAISGLRSEDEADYYC

SEQ ID NO: 105 - protein, 32 aa, Artificial Sequence
  VL(lambda)_FR3
GIPDRFSGSKSGTSATLGITGLQTGDEADYYC

SEQ ID NO: 106 - protein, 32 aa, Artificial Sequence
  VL(lambda)_FR3
GVSNRFSGSKSGNTASLTISGLQAEDEADYYC

SEQ ID NO: 107 - protein, 32 aa, Artificial Sequence
  VL(lambda)_FR3
GVPDRFSGSKSGNTASLTISGLQAEDEADYYC

SEQ ID NO: 108 - protein, 32 aa, Artificial Sequence
  VL(lambda)_FR3
GIPERFSGSNSGNTATLTISGTQAMDEADYYC

SEQ ID NO: 109 - protein, 117 aa, Artificial Sequence
  VH
QVQLVQSGAEVKKPGSSVKVSCKASGGTFSSYAMSWVRQAPGQGLEWMGTITGSGGSTYYADSVKGRVTITADESTSTAYMELSSLRSEDTAVYYCARYKSGAFDIWGQGTLVTVSS

SEQ ID NO: 110 - protein, 117 aa, Artificial Sequence
  VH
QVQLVQSGAEVKKPGSSVKVSCKASGGTFSAYYMIWVRQAPGQGLEWMGITNPSGGVTSYAQKFQGRVTITADESTSTAYMELSSLRSEDTAVYYCARVGGRGFDIWGQGTLVTVSS

SEQ ID NO: 111 - protein, 116 aa, Artificial Sequence
  VH
QVQLVQSGAEVKKPGSSVKVSCKASGGTFSTYYMTWVRQAPGQGLEWMGIINPRGGSTSYAEKFQGRVTITADESTSTAYMELSSLRSEDTAVYYCARWNLGMDVWGQGTLVTVSS

SEQ ID NO: 112 - protein, 116 aa, Artificial Sequence
  VH
QVQLVQSGAEVKKPGSSVKVSCKASGGTFSSYGMHWVRQAPGQGLEWMGAISGSGTSIYYANSVKSRVTITADESTSTAYMELSSLRSEDTAVYYCARGAGWFDYWGQGTLVTVSS

SEQ ID NO: 113 - protein, 119 aa, Artificial Sequence
  VH
QVQLVQSGAEVKKPGSSVKVSCKASGGTFSTYAMSWVRQAPGQGLEWMGAISGSGGSTLYADSVKGRVTITADESTSTAYMELSSLRSEDTAVYYCARGGSATWAFDLWGQGTLVTVSS

SEQ ID NO: 114 - protein, 116 aa, Artificial Sequence
  VH
QVQLVQSGAEVKKPGSSVKVSCKASGGTFSTYAISWVRQAPGQGLEWMGSIYYSGYTHYNPSLESRVTITADESTSTAYMELSSLRSEDTAVYYCARGYSGWMDVWGQGTLVTVSS

SEQ ID NO: 115 - protein, 117 aa, Artificial Sequence
  VH
QVQLVQSGAEVKKPGSSVKVSCKASGGTFSNFWIGWVRQAPGQGLEWMGWINPPGSITSYADSVKGRVTITADESTSTAYMELSSLRSEDTAVYYCAREVGDVLDYWGQGTLVTVSS

SEQ ID NO: 116 - protein, 116 aa, Artificial Sequence
  VH
QVQLVQSGAEVKKPGSSVKVSCKASGGTFSSYWISWVRQAPGQGLEWMGEISPSGNTNYNPSLKSRVTITADESTSTAYMELSSLRSEDTAVYYCAREVGDLLDYWGQGTLVTVSS

SEQ ID NO: 117 - protein, 118 aa, Artificial Sequence
  VH
QVQLVQSGAEVKKPGSSVKVSCKASGGTFSSYAMNWVRQAPGQGLEWMGTISGSGGQTYYADSVKGRVTITADESTSTAYMELSSLRSEDTAVYYCARWRSIWAFDYWGQGTLVTVSS

SEQ ID NO: 118 - protein, 117 aa, Artificial Sequence
  VH
QVQLVQSGAEVKKPGASVKVSCKASGYTFTSYAMSWVRQAPGQGLEWMGTITGSGGSTYYADSVKGRVTMTTDTSTSTAYMELRSLRSDDTAVYYCARYKSGAFDIWGQGTLVTVSS

SEQ ID NO: 119 - protein, 117 aa, Artificial Sequence
  VH
QVQLVQSGAEVKKPGASVKVSCKASGYTFTAYYMIWVRQAPGQGLEWMGITNPSGGVTSYAQKFQGRVTMTTDTSTSTAYMELRSLRSDDTAVYYCARVGGRGFDIWGQGTLVTVSS

SEQ ID NO: 120 - protein, 116 aa, Artificial Sequence
  VH
QVQLVQSGAEVKKPGASVKVSCKASGYTFTTYYMTWVRQAPGQGLEWMGIINPRGGSTSYAEKFQGRVTMTTDTSTSTAYMELRSLRSDDTAVYYCARWNLGMDVWGQGTLVTVSS

SEQ ID NO: 121 - protein, 116 aa, Artificial Sequence
  VH
QVQLVQSGAEVKKPGASVKVSCKASGYTFTSYGMHWVRQAPGQGLEWMGAISGSGTSIYYANSVKSRVTMTTDTSTSTAYMELRSLRSDDTAVYYCARGAGWFDYWGQGTLVTVSS

SEQ ID NO: 122 - protein, 119 aa, Artificial Sequence
  VH
QVQLVQSGAEVKKPGASVKVSCKASGYTFTTYAMSWVRQAPGQGLEWMGAISGSGGSTLYADSVKGRVTMTTDTSTSTAYMELRSLRSDDTAVYYCARGGSATWAFDLWGQGTLVTVSS

SEQ ID NO: 123 - protein, 116 aa, Artificial Sequence
  VH
QVQLVQSGAEVKKPGASVKVSCKASGYTFTTYAISWVRQAPGQGLEWMGSIYYSGYTHYNPSLESRVTMTTDTSTSTAYMELRSLRSDDTAVYYCARGYSGWMDVWGQGTLVTVSS

SEQ ID NO: 124 - protein, 117 aa, Artificial Sequence
  VH
QVQLVQSGAEVKKPGASVKVSCKASGYTFTNFWIGWVRQAPGQGLEWMGWINPPGSITSYADSVKGRVTMTTDTSTSTAYMELRSLRSDDTAVYYCAREVGDVLDYWGQGTLVTVSS

SEQ ID NO: 125 - protein, 116 aa, Artificial Sequence
  VH
QVQLVQSGAEVKKPGASVKVSCKASGYTFTSYWISWVRQAPGQGLEWMGEISPSGNTNYNPSLKSRVTMTTDTSTSTAYMELRSLRSDDTAVYYCAREVGDLLDYWGQGTLVTVSS

SEQ ID NO: 126 - protein, 118 aa, Artificial Sequence
  VH
QVQLVQSGAEVKKPGASVKVSCKASGYTFTSYAMNWVRQAPGQGLEWMGTISGSGGQTYYADSVKGRVTMTTDTSTSTAYMELRSLRSDDTAVYYCARWRSIWAFDYWGQGTLVTVSS

SEQ ID NO: 127 - protein, 117 aa, Artificial Sequence
  VH
EVQLLESGGGLVQPGGSLRLSCAASGFTFSSYAMSWVRQAPGKGLEWVSTITGSGGSTYYADSVKGRFTISRDNSKNTLYLQMNSLRAEDTAVYYCARYKSGAFDIWGQGTLVTVSS

SEQ ID NO: 128 - protein, 117 aa, Artificial Sequence
  VH
EVQLLESGGGLVQPGGSLRLSCAASGFTFSAYYMIWVRQAPGKGLEWVSITNPSGGVTSYAQKFQGRFTISRDNSKNTLYLQMNSLRAEDTAVYYCARVGGRGFDIWGQGTLVTVSS

SEQ ID NO: 129 - protein, 116 aa, Artificial Sequence
  VH
EVQLLESGGGLVQPGGSLRLSCAASGFTFSTYYMTWVRQAPGKGLEWVSIINPRGGSTSYAEKFQGRFTISRDNSKNTLYLQMNSLRAEDTAVYYCARWNLGMDVWGQGTLVTVSS

SEQ ID NO: 130 - protein, 116 aa, Artificial Sequence
  VH
EVQLLESGGGLVQPGGSLRLSCAASGFTFSSYGMHWVRQAPGKGLEWVSAISGSGTSIYYANSVKSRFTISRDNSKNTLYLQMNSLRAEDTAVYYCARGAGWFDYWGQGTLVTVSS

SEQ ID NO: 131 - protein, 119 aa, Artificial Sequence
  VH
EVQLLESGGGLVQPGGSLRLSCAASGFTFSTYAMSWVRQAPGKGLEWVSAISGSGGSTLYADSVKGRFTISRDNSKNTLYLQMNSLRAEDTAVYYCARGGSATWAFDLWGQGTLVTVSS

SEQ ID NO: 132 - protein, 116 aa, Artificial Sequence
  VH
EVQLLESGGGLVQPGGSLRLSCAASGFTFSTYAISWVRQAPGKGLEWVSSIYYSGYTHYNPSLESRFTISRDNSKNTLYLQMNSLRAEDTAVYYCARGYSGWMDVWGQGTLVTVSS

SEQ ID NO: 133 - protein, 117 aa, Artificial Sequence
  VH
EVQLLESGGGLVQPGGSLRLSCAASGFTFSNFWIGWVRQAPGKGLEWVSWINPPGSITSYADSVKGRFTISRDNSKNTLYLQMNSLRAEDTAVYYCAREVGDVLDYWGQGTLVTVSS

SEQ ID NO: 134 - protein, 116 aa, Artificial Sequence
  VH
EVQLLESGGGLVQPGGSLRLSCAASGFTFSSYWISWVRQAPGKGLEWVSEISPSGNTNYNPSLKSRFTISRDNSKNTLYLQMNSLRAEDTAVYYCAREVGDLLDYWGQGTLVTVSS

SEQ ID NO: 135 - protein, 118 aa, Artificial Sequence
  VH
EVQLLESGGGLVQPGGSLRLSCAASGFTFSSYAMNWVRQAPGKGLEWVSTISGSGGQTYYADSVKGRFTISRDNSKNTLYLQMNSLRAEDTAVYYCARWRSIWAFDYWGQGTLVTVSS

SEQ ID NO: 136 - protein, 117 aa, Artificial Sequence
  VH
EVQLLESGGGLVQPGGSLRLSCAASGFTFSSYAMSWVRQAPGKGLEWVSTITGSGGSTYYADSVKGRFTISRDNSKDTLYLQMNSLRAEDTAVYYCARYKSGAFDIWGQGTLVTVSS

SEQ ID NO: 137 - protein, 117 aa, Artificial Sequence
  VH
EVQLLESGGGLVQPGGSLRLSCAASGFTFSAYYMIWVRQAPGKGLEWVSITNPSGGVTSYAQKFQGRFTISRDNSKDTLYLQMNSLRAEDTAVYYCARVGGRGFDIWGQGTLVTVSS

SEQ ID NO: 138 - protein, 116 aa, Artificial Sequence
  VH
EVQLLESGGGLVQPGGSLRLSCAASGFTFSTYYMTWVRQAPGKGLEWVSIINPRGGSTSYAEKFQGRFTISRDNSKDTLYLQMNSLRAEDTAVYYCARWNLGMDVWGQGTLVTVSS

SEQ ID NO: 139 - protein, 116 aa, Artificial Sequence
  VH
EVQLLESGGGLVQPGGSLRLSCAASGFTFSSYGMHWVRQAPGKGLEWVSAISGSGTSIYYANSVKSRFTISRDNSKDTLYLQMNSLRAEDTAVYYCARGAGWFDYWGQGTLVTVSS

SEQ ID NO: 140 - protein, 119 aa, Artificial Sequence
  VH
EVQLLESGGGLVQPGGSLRLSCAASGFTFSTYAMSWVRQAPGKGLEWVSAISGSGGSTLYADSVKGRFTISRDNSKDTLYLQMNSLRAEDTAVYYCARGGSATWAFDLWGQGTLVTVSS

SEQ ID NO: 141 - protein, 116 aa, Artificial Sequence
  VH
EVQLLESGGGLVQPGGSLRLSCAASGFTFSTYAISWVRQAPGKGLEWVSSIYYSGYTHYNPSLESRFTISRDNSKDTLYLQMNSLRAEDTAVYYCARGYSGWMDVWGQGTLVTVSS

SEQ ID NO: 142 - protein, 117 aa, Artificial Sequence
  VH
EVQLLESGGGLVQPGGSLRLSCAASGFTFSNFWIGWVRQAPGKGLEWVSWINPPGSITSYADSVKGRFTISRDNSKDTLYLQMNSLRAEDTAVYYCAREVGDVLDYWGQGTLVTVSS

SEQ ID NO: 143 - protein, 116 aa, Artificial Sequence
  VH
EVQLLESGGGLVQPGGSLRLSCAASGFTFSSYWISWVRQAPGKGLEWVSEISPSGNTNYNPSLKSRFTISRDNSKDTLYLQMNSLRAEDTAVYYCAREVGDLLDYWGQGTLVTVSS

SEQ ID NO: 144 - protein, 118 aa, Artificial Sequence
  VH
EVQLLESGGGLVQPGGSLRLSCAASGFTFSSYAMNWVRQAPGKGLEWVSTISGSGGQTYYADSVKGRFTISRDNSKDTLYLQMNSLRAEDTAVYYCARWRSIWAFDYWGQGTLVTVSS

SEQ ID NO: 145 - protein, 117 aa, Artificial Sequence
  VH
EVQLLESGGGLVQPGSSLRLSCAASGFTFSSYAMSWVRQAPGKGLEWVSTITGSGGSTYYADSVKGRFTISRDNSKNTLYLQMNSLRAEDTAVYYCARYKSGAFDIWGQGTLVTVSS

SEQ ID NO: 146 - protein, 117 aa, Artificial Sequence
  VH
EVQLLESGGGLVQPGSSLRLSCAASGFTFSAYYMIWVRQAPGKGLEWVSITNPSGGVTSYAQKFQGRFTISRDNSKNTLYLQMNSLRAEDTAVYYCARVGGRGFDIWGQGTLVTVSS

SEQ ID NO: 147 - protein, 116 aa, Artificial Sequence
  VH
EVQLLESGGGLVQPGSSLRLSCAASGFTFSTYYMTWVRQAPGKGLEWVSIINPRGGSTSYAEKFQGRFTISRDNSKNTLYLQMNSLRAEDTAVYYCARWNLGMDVWGQGTLVTVSS

SEQ ID NO: 148 - protein, 116 aa, Artificial Sequence
  VH
EVQLLESGGGLVQPGSSLRLSCAASGFTFSSYGMHWVRQAPGKGLEWVSAISGSGTSIYYANSVKSRFTISRDNSKNTLYLQMNSLRAEDTAVYYCARGAGWFDYWGQGTLVTVSS

SEQ ID NO: 149 - protein, 119 aa, Artificial Sequence
  VH
EVQLLESGGGLVQPGSSLRLSCAASGFTFSTYAMSWVRQAPGKGLEWVSAISGSGGSTLYADSVKGRFTISRDNSKNTLYLQMNSLRAEDTAVYYCARGGSATWAFDLWGQGTLVTVSS

SEQ ID NO: 150 - protein, 116 aa, Artificial Sequence
  VH
EVQLLESGGGLVQPGSSLRLSCAASGFTFSTYAISWVRQAPGKGLEWVSSIYYSGYTHYNPSLESRFTISRDNSKNTLYLQMNSLRAEDTAVYYCARGYSGWMDVWGQGTLVTVSS

SEQ ID NO: 151 - protein, 117 aa, Artificial Sequence
  VH
EVQLLESGGGLVQPGSSLRLSCAASGFTFSNFWIGWVRQAPGKGLEWVSWINPPGSITSYADSVKGRFTISRDNSKNTLYLQMNSLRAEDTAVYYCAREVGDVLDYWGQGTLVTVSS

SEQ ID NO: 152 - protein, 116 aa, Artificial Sequence
  VH
EVQLLESGGGLVQPGSSLRLSCAASGFTFSSYWISWVRQAPGKGLEWVSEISPSGNTNYNPSLKSRFTISRDNSKNTLYLQMNSLRAEDTAVYYCAREVGDLLDYWGQGTLVTVSS

SEQ ID NO: 153 - protein, 118 aa, Artificial Sequence
  VH
EVQLLESGGGLVQPGSSLRLSCAASGFTFSSYAMNWVRQAPGKGLEWVSTISGSGGQTYYADSVKGRFTISRDNSKNTLYLQMNSLRAEDTAVYYCARWRSIWAFDYWGQGTLVTVSS

SEQ ID NO: 154 - protein, 117 aa, Artificial Sequence
  VH
EVQLVESGGGLVQPGGSLRLSCAASGFTFSSYAMSWVRQAPGKGLEWVSTITGSGGSTYYADSVKGRFTISRDNAKNSLYLQMNSLRDEDTAVYYCARYKSGAFDIWGQGTLVTVSS

SEQ ID NO: 155 - protein, 117 aa, Artificial Sequence
  VH
EVQLVESGGGLVQPGGSLRLSCAASGFTFSAYYMIWVRQAPGKGLEWVSITNPSGGVTSYAQKFQGRFTISRDNAKNSLYLQMNSLRDEDTAVYYCARVGGRGFDIWGQGTLVTVSS

SEQ ID NO: 156 - protein, 116 aa, Artificial Sequence
  VH
EVQLVESGGGLVQPGGSLRLSCAASGFTFSTYYMTWVRQAPGKGLEWVSIINPRGGSTSYAEKFQGRFTISRDNAKNSLYLQMNSLRDEDTAVYYCARWNLGMDVWGQGTLVTVSS

SEQ ID NO: 157 - protein, 116 aa, Artificial Sequence
  VH
EVQLVESGGGLVQPGGSLRLSCAASGFTFSSYGMHWVRQAPGKGLEWVSAISGSGTSIYYANSVKSRFTISRDNAKNSLYLQMNSLRDEDTAVYYCARGAGWFDYWGQGTLVTVSS

SEQ ID NO: 158 - protein, 119 aa, Artificial Sequence
  VH
EVQLVESGGGLVQPGGSLRLSCAASGFTFSTYAMSWVRQAPGKGLEWVSAISGSGGSTLYADSVKGRFTISRDNAKNSLYLQMNSLRDEDTAVYYCARGGSATWAFDLWGQGTLVTVSS

SEQ ID NO: 159 - protein, 116 aa, Artificial Sequence
  VH
EVQLVESGGGLVQPGGSLRLSCAASGFTFSTYAISWVRQAPGKGLEWVSSIYYSGYTHYNPSLESRFTISRDNAKNSLYLQMNSLRDEDTAVYYCARGYSGWMDVWGQGTLVTVSS

SEQ ID NO: 160 - protein, 117 aa, Artificial Sequence
  VH
EVQLVESGGGLVQPGGSLRLSCAASGFTFSNFWIGWVRQAPGKGLEWVSWINPPGSITSYADSVKGRFTISRDNAKNSLYLQMNSLRDEDTAVYYCAREVGDVLDYWGQGTLVTVSS

SEQ ID NO: 161 - protein, 116 aa, Artificial Sequence
  VH
EVQLVESGGGLVQPGGSLRLSCAASGFTFSSYWISWVRQAPGKGLEWVSEISPSGNTNYNPSLKSRFTISRDNAKNSLYLQMNSLRDEDTAVYYCAREVGDLLDYWGQGTLVTVSS

SEQ ID NO: 162 - protein, 118 aa, Artificial Sequence
  VH
EVQLVESGGGLVQPGGSLRLSCAASGFTFSSYAMNWVRQAPGKGLEWVSTISGSGGQTYYADSVKGRFTISRDNAKNSLYLQMNSLRDEDTAVYYCARWRSIWAFDYWGQGTLVTVSS

SEQ ID NO: 163 - protein, 117 aa, Artificial Sequence
  VH
QVQLQQWGAGLLKPSETLSLTCAVYGGSFSSYAMSWIRQPPGKGLEWIGTITGSGGSTYYADSVKGRVTISVDTSKNQFSLKLSSVTAADTAVYYCARYKSGAFDIWGQGTLVTVSS

SEQ ID NO: 164 - protein, 117 aa, Artificial Sequence
  VH
QVQLQQWGAGLLKPSETLSLTCAVYGGSFSAYYMIWIRQPPGKGLEWIGITNPSGGVTSYAQKFQGRVTISVDTSKNQFSLKLSSVTAADTAVYYCARVGGRGFDIWGQGTLVTVSS

SEQ ID NO: 165 - protein, 116 aa, Artificial Sequence
  VH
QVQLQQWGAGLLKPSETLSLTCAVYGGSFSTYYMTWIRQPPGKGLEWIGIINPRGGSTSYAEKFQGRVTISVDTSKNQFSLKLSSVTAADTAVYYCARWNLGMDVWGQGTLVTVSS

SEQ ID NO: 166 - protein, 116 aa, Artificial Sequence
  VH
QVQLQQWGAGLLKPSETLSLTCAVYGGSFSSYGMHWIRQPPGKGLEWIGAISGSGTSIYYANSVKSRVTISVDTSKNQFSLKLSSVTAADTAVYYCARGAGWFDYWGQGTLVTVSS

SEQ ID NO: 167 - protein, 119 aa, Artificial Sequence
  VH
QVQLQQWGAGLLKPSETLSLTCAVYGGSFSTYAMSWIRQPPGKGLEWIGAISGSGGSTLYADSVKGRVTISVDTSKNQFSLKLSSVTAADTAVYYCARGGSATWAFDLWGQGTLVTVSS

SEQ ID NO: 168 - protein, 116 aa, Artificial Sequence
  VH
QVQLQQWGAGLLKPSETLSLTCAVYGGSFSTYAISWIRQPPGKGLEWIGSIYYSGYTHYNPSLESRVTISVDTSKNQFSLKLSSVTAADTAVYYCARGYSGWMDVWGQGTLVTVSS

SEQ ID NO: 169 - protein, 117 aa, Artificial Sequence
  VH
QVQLQQWGAGLLKPSETLSLTCAVYGGSFSNFWIGWIRQPPGKGLEWIGWINPPGSITSYADSVKGRVTISVDTSKNQFSLKLSSVTAADTAVYYCAREVGDVLDYWGQGTLVTVSS

SEQ ID NO: 170 - protein, 116 aa, Artificial Sequence
  VH
QVQLQQWGAGLLKPSETLSLTCAVYGGSFSSYWISWIRQPPGKGLEWIGEISPSGNTNYNPSLKSRVTISVDTSKNQFSLKLSSVTAADTAVYYCAREVGDLLDYWGQGTLVTVSS

SEQ ID NO: 171 - protein, 118 aa, Artificial Sequence
  VH
QVQLQQWGAGLLKPSETLSLTCAVYGGSFSSYAMNWIRQPPGKGLEWIGTISGSGGQTYYADSVKGRVTISVDTSKNQFSLKLSSVTAADTAVYYCARWRSIWAFDYWGQGTLVTVSS

SEQ ID NO: 172 - protein, 117 aa, Artificial Sequence
  VH
QVQLQESGPGLVKPSETLSLTCTVSGGSISSYAMSWIRQPPGKGLEWIGTITGSGGSTYYADSVKGRVTISVDTSKNQFSLKLSSVTAADTAVYYCARYKSGAFDIWGQGTLVTVSS

SEQ ID NO: 173 - protein, 117 aa, Artificial Sequence
  VH
QVQLQESGPGLVKPSETLSLTCTVSGGSISAYYMIWIRQPPGKGLEWIGITNPSGGVTSYAQKFQGRVTISVDTSKNQFSLKLSSVTAADTAVYYCARVGGRGFDIWGQGTLVTVSS

SEQ ID NO: 174 - protein, 116 aa, Artificial Sequence
  VH
QVQLQESGPGLVKPSETLSLTCTVSGGSISTYYMTWIRQPPGKGLEWIGIINPRGGSTSYAEKFQGRVTISVDTSKNQFSLKLSSVTAADTAVYYCARWNLGMDVWGQGTLVTVSS

SEQ ID NO: 175 - protein, 116 aa, Artificial Sequence
  VH
QVQLQESGPGLVKPSETLSLTCTVSGGSISSYGMHWIRQPPGKGLEWIGAISGSGTSIYYANSVKSRVTISVDTSKNQFSLKLSSVTAADTAVYYCARGAGWFDYWGQGTLVTVSS

SEQ ID NO: 176 - protein, 119 aa, Artificial Sequence
  VH
QVQLQESGPGLVKPSETLSLTCTVSGGSISTYAMSWIRQPPGKGLEWIGAISGSGGSTLYADSVKGRVTISVDTSKNQFSLKLSSVTAADTAVYYCARGGSATWAFDLWGQGTLVTVSS

SEQ ID NO: 177 - protein, 116 aa, Artificial Sequence
  VH
QVQLQESGPGLVKPSETLSLTCTVSGGSISTYAISWIRQPPGKGLEWIGSIYYSGYTHYNPSLESRVTISVDTSKNQFSLKLSSVTAADTAVYYCARGYSGWMDVWGQGTLVTVSS

SEQ ID NO: 178 - protein, 117 aa, Artificial Sequence
  VH
QVQLQESGPGLVKPSETLSLTCTVSGGSISNFWIGWIRQPPGKGLEWIGWINPPGSITSYADSVKGRVTISVDTSKNQFSLKLSSVTAADTAVYYCAREVGDVLDYWGQGTLVTVSS

SEQ ID NO: 179 - protein, 116 aa, Artificial Sequence
  VH
QVQLQESGPGLVKPSETLSLTCTVSGGSISSYWISWIRQPPGKGLEWIGEISPSGNTNYNPSLKSRVTISVDTSKNQFSLKLSSVTAADTAVYYCAREVGDLLDYWGQGTLVTVSS

SEQ ID NO: 180 - protein, 118 aa, Artificial Sequence
  VH
QVQLQESGPGLVKPSETLSLTCTVSGGSISSYAMNWIRQPPGKGLEWIGTISGSGGQTYYADSVKGRVTISVDTSKNQFSLKLSSVTAADTAVYYCARWRSIWAFDYWGQGTLVTVSS

SEQ ID NO: 181 - protein, 117 aa, Artificial Sequence
  VH
EVQLVQSGAEVKKPGESLKISCKGSGYSFTSYAMSWVRQMPGKGLEWMGTITGSGGSTYYADSVKGQVTISADKSISTAYLQWSSLKASDTAMYYCARYKSGAFDIWGQGTLVTVSS

SEQ ID NO: 182 - protein, 117 aa, Artificial Sequence
  VH
EVQLVQSGAEVKKPGESLKISCKGSGYSFTAYYMIWVRQMPGKGLEWMGITNPSGGVTSYAQKFQGQVTISADKSISTAYLQWSSLKASDTAMYYCARVGGRGFDIWGQGTLVTVSS

SEQ ID NO: 183 - protein, 116 aa, Artificial Sequence
  VH
EVQLVQSGAEVKKPGESLKISCKGSGYSFTTYYMTWVRQMPGKGLEWMGIINPRGGSTSYAEKFQGQVTISADKSISTAYLQWSSLKASDTAMYYCARWNLGMDVWGQGTLVTVSS

SEQ ID NO: 184 - protein, 116 aa, Artificial Sequence
  VH
EVQLVQSGAEVKKPGESLKISCKGSGYSFTSYGMHWVRQMPGKGLEWMGAISGSGTSIYYANSVKSQVTISADKSISTAYLQWSSLKASDTAMYYCARGAGWFDYWGQGTLVTVSS

SEQ ID NO: 185 - protein, 119 aa, Artificial Sequence
  VH
EVQLVQSGAEVKKPGESLKISCKGSGYSFTTYAMSWVRQMPGKGLEWMGAISGSGGSTLYADSVKGQVTISADKSISTAYLQWSSLKASDTAMYYCARGGSATWAFDLWGQGTLVTVSS

SEQ ID NO: 186 - protein, 116 aa, Artificial Sequence
  VH
EVQLVQSGAEVKKPGESLKISCKGSGYSFTTYAISWVRQMPGKGLEWMGSIYYSGYTHYNPSLESQVTISADKSISTAYLQWSSLKASDTAMYYCARGYSGWMDVWGQGTLVTVSS

SEQ ID NO: 187 - protein, 117 aa, Artificial Sequence
  VH
EVQLVQSGAEVKKPGESLKISCKGSGYSFTNFWIGWVRQMPGKGLEWMGWINPPGSITSYADSVKGQVTISADKSISTAYLQWSSLKASDTAMYYCAREVGDVLDYWGQGTLVTVSS

SEQ ID NO: 188 - protein, 116 aa, Artificial Sequence
  VH
EVQLVQSGAEVKKPGESLKISCKGSGYSFTSYWISWVRQMPGKGLEWMGEISPSGNTNYNPSLKSQVTISADKSISTAYLQWSSLKASDTAMYYCAREVGDLLDYWGQGTLVTVSS

SEQ ID NO: 189 - protein, 118 aa, Artificial Sequence
  VH
EVQLVQSGAEVKKPGESLKISCKGSGYSFTSYAMNWVRQMPGKGLEWMGTISGSGGQTYYADSVKGQVTISADKSISTAYLQWSSLKASDTAMYYCARWRSIWAFDYWGQGTLVTVSS

SEQ ID NO: 190 - protein, 119 aa, Artificial Sequence
  VH
QVQLQQSGPGLVKPSQTLSLTCAISGDSVSSYAMSWNWIRQSPSRGLEWLGTITGSGGSTYYADSVKGRITINPDTSKNQFSLQLNSVTPEDTAVYYCARYKSGAFDIWGQGTLVTVSS

SEQ ID NO: 191 - protein, 119 aa, Artificial Sequence
  VH
QVQLQQSGPGLVKPSQTLSLTCAISGDSVSAYYMIWNWIRQSPSRGLEWLGITNPSGGVTSYAQKFQGRITINPDTSKNQFSLQLNSVTPEDTAVYYCARVGGRGFDIWGQGTLVTVSS

SEQ ID NO: 192 - protein, 118 aa, Artificial Sequence
  VH
QVQLQQSGPGLVKPSQTLSLTCAISGDSVSTYYMTWNWIRQSPSRGLEWLGIINPRGGSTSYAEKFQGRITINPDTSKNQFSLQLNSVTPEDTAVYYCARWNLGMDVWGQGTLVTVSS

SEQ ID NO: 193 - protein, 118 aa, Artificial Sequence
  VH
QVQLQQSGPGLVKPSQTLSLTCAISGDSVSSYGMHWNWIRQSPSRGLEWLGAISGSGTSIYYANSVKSRITINPDTSKNQFSLQLNSVTPEDTAVYYCARGAGWFDYWGQGTLVTVSS

SEQ ID NO: 194 - protein, 121 aa, Artificial Sequence
  VH
QVQLQQSGPGLVKPSQTLSLTCAISGDSVSTYAMSWNWIRQSPSRGLEWLGAISGSGGSTLYADSVKGRITINPDTSKNQFSLQLNSVTPEDTAVYYCARGGSATWAFDLWGQGTLVTVSS

SEQ ID NO: 195 - protein, 118 aa, Artificial Sequence
  VH
QVQLQQSGPGLVKPSQTLSLTCAISGDSVSTYAISWNWIRQSPSRGLEWLGSIYYSGYTHYNPSLESRITINPDTSKNQFSLQLNSVTPEDTAVYYCARGYSGWMDVWGQGTLVTVSS

SEQ ID NO: 196 - protein, 120 aa, Artificial Sequence
  VH
QVQLQQSGPGLVKPSQTLSLTCAISGDSVSSYAMNWNWIRQSPSRGLEWLGTISGSGGQTYYADSVKGRITINPDTSKNQFSLQLNSVTPEDTAVYYCARWRSIWAFDYWGQGTLVTVSS

SEQ ID NO: 197 - protein, 118 aa, Artificial Sequence
  VH
QVQLQQSGPGLVKPSQTLSLTCAISGDSVSSYWISWNWIRQSPSRGLEWLGEISPSGNTNYNPSLKSRITINPDTSKNQFSLQLNSVTPEDTAVYYCAREVGDLLDYWGQGTLVTVSS

SEQ ID NO: 198 - protein, 120 aa, Artificial Sequence; identical to SEQ ID NO: 196
  VH

SEQ ID NO: 199 - protein, 107 aa, Artificial Sequence
  VL
QSVLTQPPSASGTPGQRVTISCRASQGIGNYLGWYQQLPGTAPKLLIYTASSLQSGVPDRFSGSKSGTSASLAISGLQSEDEADYYCQQGYSTPSWTFGQGTKVEIK

SEQ ID NO: 200 - protein, 106 aa, Artificial Sequence
  VL
QSVLTQPPSASGTPGQRVTISCHASQGISNYVAWYQQLPGTAPKLLIYDASNRATGVPDRFSGSKSGTSASLAISGLQSEDEADYYCQQSYSTLLTFGQGTKVEIK

SEQ ID NO: 201 - protein, 106 aa, Artificial Sequence
  VL
QSVLTQPPSASGTPGQRVTISCRVSQSISSYLNWYQQLPGTAPKLLIYDASHRATGVPDRFSGSKSGTSASLAISGLQSEDEADYYCQQYYAPSVTFGQGTKVEIK

SEQ ID NO: 202 - protein, 110 aa, Artificial Sequence
  VL
QSVLTQPPSASGTPGQRVTISCSGSTSNIGSNTVNWYQQLPGTAPKLLIYGKNIRPNGVPDRFSGSKSGTSASLAISGLQSEDEADYYCQSYDSSLSGWVFGGGTKLTVL

SEQ ID NO: 203 - protein, 110 aa, Artificial Sequence
  VL
QSVLTQPPSASGTPGQRVTISCSGSSSNIGSNYVYWYQQLPGTAPKLLIYRNNQRPSGVPDRFSGSKSGTSASLAISGLQSEDEADYYCGTWDSSLSGVVFGGGTKLTVL

SEQ ID NO: 204 - protein, 108 aa, Artificial Sequence
  VL
QSVLTQPPSASGTPGQRVTISCGGNNIGSKSVHWYQQLPGTAPKLLIYDNNKRPSGVPDRFSGSKSGTSASLAISGLQSEDEADYYCAAWDDSLNVPVFGGGTKLTVL

SEQ ID NO: 205 - protein, 111 aa, Artificial Sequence
  VL
QSVLTQPPSASGTPGQRVTISCTGTSSDIGRYNYVSWYQQLPGTAPKLLIYRNTQRPSGVPDRFSGSKSGTSASLAISGLQSEDEADYYCGTWDSSLNAYVFGGGTKLTVL

SEQ ID NO: 206 - protein, 108 aa, Artificial Sequence
  VL
QSVLTQPPSASGTPGQRVTISCSGSNIGNNYVSWYQQLPGTAPKLLIYQDSKRPSGVPDRFSGSKSGTSASLAISGLQSEDEADYYCLVWDRSSDHFVFGGGTKLTVL

SEQ ID NO: 207 - protein, 108 aa, Artificial Sequence
  VL
QSVLTQPPSASGTPGQRVTISCSDNNIGSKSVHWYQQLPGTAPKLLIYDNNKRPSGVPDRFSGSKSGTSASLAISGLQSEDEADYYCGTWDDSLSGPVFGGGTKLTVL

SEQ ID NO: 208 - protein, 107 aa, Artificial Sequence
  VL
QSVLTQPPSASGTPGQRVTISCRASQGIGNYLGWYQQLPGTAPKLLIYTASSLQSGVPDRFSGSKSGTSASLAISGLHSEDEADYYCQQGYSTPSWTFGQGTKVEIK

SEQ ID NO: 209 - protein, 106 aa, Artificial Sequence
  VL
QSVLTQPPSASGTPGQRVTISCHASQGISNYVAWYQQLPGTAPKLLIYDASNRATGVPDRFSGSKSGTSASLAISGLHSEDEADYYCQQSYSTLLTFGQGTKVEIK

SEQ ID NO: 210 - protein, 106 aa, Artificial Sequence
  VL
QSVLTQPPSASGTPGQRVTISCRVSQSISSYLNWYQQLPGTAPKLLIYDASHRATGVPDRFSGSKSGTSASLAISGLHSEDEADYYCQQYYAPSVTFGQGTKVEIK

SEQ ID NO: 211 - protein, 110 aa, Artificial Sequence
  VL
QSVLTQPPSASGTPGQRVTISCSGSTSNIGSNTVNWYQQLPGTAPKLLIYGKNIRPNGVPDRFSGSKSGTSASLAISGLHSEDEADYYCQSYDSSLSGWVFGGGTKLTVL

SEQ ID NO: 212 - protein, 110 aa, Artificial Sequence
  VL
QSVLTQPPSASGTPGQRVTISCSGSSSNIGSNYVYWYQQLPGTAPKLLIYRNNQRPSGVPDRFSGSKSGTSASLAISGLHSEDEADYYCGTWDSSLSGVVFGGGTKLTVL

SEQ ID NO: 213 - protein, 108 aa, Artificial Sequence
  VL
QSVLTQPPSASGTPGQRVTISCGGNNIGSKSVHWYQQLPGTAPKLLIYDNNKRPSGVPDRFSGSKSGTSASLAISGLHSEDEADYYCAAWDDSLNVPVFGGGTKLTVL

SEQ ID NO: 214 - protein, 111 aa, Artificial Sequence
  VL
QSVLTQPPSASGTPGQRVTISCTGTSSDIGRYNYVSWYQQLPGTAPKLLIYRNTQRPSGVPDRFSGSKSGTSASLAISGLHSEDEADYYCGTWDSSLNAYVFGGGTKLTVL

SEQ ID NO: 215 - protein, 108 aa, Artificial Sequence
  VL
QSVLTQPPSASGTPGQRVTISCSGSNIGNNYVSWYQQLPGTAPKLLIYQDSKRPSGVPDRFSGSKSGTSASLAISGLHSEDEADYYCLVWDRSSDHFVFGGGTKLTVL

SEQ ID NO: 216 - protein, 108 aa, Artificial Sequence; identical to SEQ ID NO: 215
  VL

SEQ ID NO: 217 - protein, 106 aa, Artificial Sequence
  VL
QSVLTQPPSASGTPGQRVTISCRVSQSISSYLNWYQQLPGTAPKLLIYDASHRATGVPDRFSGSKSGTSASLAISGLRSEDEADYYCQQYYAPSVTFGQGTKVEIK

SEQ ID NO: 218 - protein, 106 aa, Artificial Sequence
  VL
QSVLTQPPSASGTPGQRVTISCHASQGISNYVAWYQQLPGTAPKLLIYDASNRATGVPDRFSGSKSGTSASLAISGLRSEDEADYYCQQSYSTLLTFGQGTKVEIK

SEQ ID NO: 219 - protein, 106 aa, Artificial Sequence; identical to SEQ ID NO: 217
  VL

SEQ ID NO: 220 - protein, 110 aa, Artificial Sequence
  VL
QSVLTQPPSASGTPGQRVTISCSGSTSNIGSNTVNWYQQLPGTAPKLLIYGKNIRPNGVPDRFSGSKSGTSASLAISGLRSEDEADYYCQSYDSSLSGWVFGGGTKLTVL

SEQ ID NO: 221 - protein, 110 aa, Artificial Sequence
  VL
QSVLTQPPSASGTPGQRVTISCSGSSSNIGSNYVYWYQQLPGTAPKLLIYRNNQRPSGVPDRFSGSKSGTSASLAISGLRSEDEADYYCGTWDSSLSGVVFGGGTKLTVL

SEQ ID NO: 222 - protein, 108 aa, Artificial Sequence
  VL
QSVLTQPPSASGTPGQRVTISCGGNNIGSKSVHWYQQLPGTAPKLLIYDNNKRPSGVPDRFSGSKSGTSASLAISGLRSEDEADYYCAAWDDSLNVPVFGGGTKLTVL

SEQ ID NO: 223 - protein, 111 aa, Artificial Sequence
  VL
QSVLTQPPSASGTPGQRVTISCTGTSSDIGRYNYVSWYQQLPGTAPKLLIYRNTQRPSGVPDRFSGSKSGTSASLAISGLRSEDEADYYCGTWDSSLNAYVFGGGTKLTVL

SEQ ID NO: 224 - protein, 108 aa, Artificial Sequence
  VL
QSVLTQPPSASGTPGQRVTISCSGSNIGNNYVSWYQQLPGTAPKLLIYQDSKRPSGVPDRFSGSKSGTSASLAISGLRSEDEADYYCLVWDRSSDHFVFGGGTKLTVL

SEQ ID NO: 225 - protein, 108 aa, Artificial Sequence
  VL
QSVLTQPPSASGTPGQRVTISCSDNNIGSKSVHWYQQLPGTAPKLLIYDNNKRPSGVPDRFSGSKSGTSASLAISGLRSEDEADYYCGTWDDSLSGPVFGGGTKLTVL

SEQ ID NO: 226 - protein, 107 aa, Artificial Sequence
  VL
QSVLTQPPSASGTPGQRVTISCRASQGIGNYLGWYQQLPGTAPKLLIYTASSLQSGVPNRFSGSKSGTSASLAISGLRSEDEADYYCQQGYSTPSWTFGQGTKVEIK

SEQ ID NO: 227 - protein, 106 aa, Artificial Sequence
  VL
QSVLTQPPSASGTPGQRVTISCHASQGISNYVAWYQQLPGTAPKLLIYDASNRATGVPNRFSGSKSGTSASLAISGLRSEDEADYYCQQSYSTLLTFGQGTKVEIK

SEQ ID NO: 228 - protein, 106 aa, Artificial Sequence
  VL
QSVLTQPPSASGTPGQRVTISCRVSQSISSYLNWYQQLPGTAPKLLIYDASHRATGVPNRFSGSKSGTSASLAISGLRSEDEADYYCQQYYAPSVTFGQGTKVEIK

SEQ ID NO: 229 - protein, 110 aa, Artificial Sequence
  VL
QSVLTQPPSASGTPGQRVTISCSGSTSNIGSNTVNWYQQLPGTAPKLLIYGKNIRPNGVPNRFSGSKSGTSASLAISGLRSEDEADYYCQSYDSSLSGWVFGGGTKLTVL

SEQ ID NO: 230 - protein, 110 aa, Artificial Sequence
  VL
QSVLTQPPSASGTPGQRVTISCSGSSSNIGSNYVYWYQQLPGTAPKLLIYRNNQRPSGVPNRFSGSKSGTSASLAISGLRSEDEADYYCGTWDSSLSGVVFGGGTKLTVL

SEQ ID NO: 231 - protein, 108 aa, Artificial Sequence
  VL
QSVLTQPPSASGTPGQRVTISCGGNNIGSKSVHWYQQLPGTAPKLLIYDNNKRPSGVPNRFSGSKSGTSASLAISGLRSEDEADYYCAAWDDSLNVPVFGGGTKLTVL

SEQ ID NO: 232 - protein, 111 aa, Artificial Sequence
  VL
QSVLTQPPSASGTPGQRVTISCTGTSSDIGRYNYVSWYQQLPGTAPKLLIYRNTQRPSGVPNRFSGSKSGTSASLAISGLRSEDEADYYCGTWDSSLNAYVFGGGTKLTVL

SEQ ID NO: 233 - protein, 108 aa, Artificial Sequence
  VL
QSVLTQPPSASGTPGQRVTISCSGSNIGNNYVSWYQQLPGTAPKLLIYQDSKRPSGVPNRFSGSKSGTSASLAISGLRSEDEADYYCLVWDRSSDHFVFGGGTKLTVL

SEQ ID NO: 234 - protein, 108 aa, Artificial Sequence
  VL
QSVLTQPPSASGTPGQRVTISCSDNNIGSKSVHWYQQLPGTAPKLLIYDNNKRPSGVPNRFSGSKSGTSASLAISGLRSEDEADYYCGTWDDSLSGPVFGGGTKLTVL

SEQ ID NO: 235 - protein, 107 aa, Artificial Sequence
  VL
QSVLTQPPSVSAAPGQKVTISCRASQGIGNYLGWYQQLPGTAPKLLIYTASSLQSGIPDRFSGSKSGTSATLGITGLQTGDEADYYCQQGYSTPSWTFGQGTKVEIK

SEQ ID NO: 236 - protein, 106 aa, Artificial Sequence
  VL
QSVLTQPPSVSAAPGQKVTISCHASQGISNYVAWYQQLPGTAPKLLIYDASNRATGIPDRFSGSKSGTSATLGITGLQTGDEADYYCQQSYSTLLTFGQGTKVEIK

SEQ ID NO: 237 - protein, 106 aa, Artificial Sequence
  VL
QSVLTQPPSVSAAPGQKVTISCRVSQSISSYLNWYQQLPGTAPKLLIYDASHRATGIPDRFSGSKSGTSATLGITGLQTGDEADYYCQQYYAPSVTFGQGTKVEIK

SEQ ID NO: 238 - protein, 110 aa, Artificial Sequence
  VL
QSVLTQPPSVSAAPGQKVTISCSGSTSNIGSNTVNWYQQLPGTAPKLLIYGKNIRPNGIPDRFSGSKSGTSATLGITGLQTGDEADYYCQSYDSSLSGWVFGGGTKLTVL

SEQ ID NO: 239 - protein, 110 aa, Artificial Sequence
  VL
QSVLTQPPSVSAAPGQKVTISCSGSSSNIGSNYVYWYQQLPGTAPKLLIYRNNQRPSGIPDRFSGSKSGTSATLGITGLQTGDEADYYCGTWDSSLSGVVFGGGTKLTVL

SEQ ID NO: 240 - protein, 108 aa, Artificial Sequence
  VL
QSVLTQPPSVSAAPGQKVTISCGGNNIGSKSVHWYQQLPGTAPKLLIYDNNKRPSGIPDRFSGSKSGTSATLGITGLQTGDEADYYCAAWDDSLNVPVFGGGTKLTVL

SEQ ID NO: 241 - protein, 111 aa, Artificial Sequence
  VL
QSVLTQPPSVSAAPGQKVTISCTGTSSDIGRYNYVSWYQQLPGTAPKLLIYRNTQRPSGIPDRFSGSKSGTSATLGITGLQTGDEADYYCGTWDSSLNAYVFGGGTKLTVL

SEQ ID NO: 242 - protein, 108 aa, Artificial Sequence
  VL
QSVLTQPPSVSAAPGQKVTISCSGSNIGNNYVSWYQQLPGTAPKLLIYQDSKRPSGIPDRFSGSKSGTSATLGITGLQTGDEADYYCLVWDRSSDHFVFGGGTKLTVL

SEQ ID NO: 243 - protein, 108 aa, Artificial Sequence
  VL
QSVLTQPPSVSAAPGQKVTISCSDNNIGSKSVHWYQQLPGTAPKLLIYDNNKRPSGIPDRFSGSKSGTSATLGITGLQTGDEADYYCGTWDDSLSGPVFGGGTKLTVL

SEQ ID NO: 244 - protein, 107 aa, Artificial Sequence
  VL
QSALTQPASVSGSPGQSITISCRASQGIGNYLGWYQQHPGKAPKLMIYTASSLQSGVSNRFSGSKSGNTASLTISGLQAEDEADYYCQQGYSTPSWTFGQGTKVEIK

SEQ ID NO: 245 - protein, 106 aa, Artificial Sequence
  VL
QSALTQPASVSGSPGQSITISCHASQGISNYVAWYQQHPGKAPKLMIYDASNRATGVSNRFSGSKSGNTASLTISGLQAEDEADYYCQQSYSTLLTFGQGTKVEIK

SEQ ID NO: 246 - protein, 106 aa, Artificial Sequence
  VL
QSALTQPASVSGSPGQSITISCRVSQSISSYLNWYQQHPGKAPKLMIYDASHRATGVSNRFSGSKSGNTASLTISGLQAEDEADYYCQQYYAPSVTFGQGTKVEIK

SEQ ID NO: 247 - protein, 110 aa, Artificial Sequence
  VL
QSALTQPASVSGSPGQSITISCSGSTSNIGSNTVNWYQQHPGKAPKLMIYGKNIRPNGVSNRFSGSKSGNTASLTISGLQAEDEADYYCQSYDSSLSGWVFGGGTKLTVL

SEQ ID NO: 248 - protein, 110 aa, Artificial Sequence
  VL
QSALTQPASVSGSPGQSITISCSGSSSNIGSNYVYWYQQHPGKAPKLMIYRNNQRPSGVSNRFSGSKSGNTASLTISGLQAEDEADYYCGTWDSSLSGVVFGGGTKLTVL

SEQ ID NO: 249 - protein, 108 aa, Artificial Sequence
  VL
QSALTQPASVSGSPGQSITISCGGNNIGSKSVHWYQQHPGKAPKLMIYDNNKRPSGVSNRFSGSKSGNTASLTISGLQAEDEADYYCAAWDDSLNVPVFGGGTKLTVL

SEQ ID NO: 250 - protein, 111 aa, Artificial Sequence
  VL
QSALTQPASVSGSPGQSITISCTGTSSDIGRYNYVSWYQQHPGKAPKLMIYRNTQRPSGVSNRFSGSKSGNTASLTISGLQAEDEADYYCGTWDSSLNAYVFGGGTKLTVL

SEQ ID NO: 251 - protein, 108 aa, Artificial Sequence
  VL
QSALTQPASVSGSPGQSITISCSGSNIGNNYVSWYQQHPGKAPKLMIYQDSKRPSGVSNRFSGSKSGNTASLTISGLQAEDEADYYCLVWDRSSDHFVFGGGTKLTVL

SEQ ID NO: 252 - protein, 108 aa, Artificial Sequence
  VL
QSALTQPASVSGSPGQSITISCSDNNIGSKSVHWYQQHPGKAPKLMIYDNNKRPSGVSNRFSGSKSGNTASLTISGLQAEDEADYYCGTWDDSLSGPVFGGGTKLTVL

SEQ ID NO: 253 - protein, 107 aa, Artificial Sequence
  VL
QSALTQPRSVSGSPGQSVTISCRASQGIGNYLGWYQQHPGKAPKLMIYTASSLQSGVPDRFSGSKSGNTASLTISGLQAEDEADYYCQQGYSTPSWTFGQGTKVEIK

SEQ ID NO: 254 - protein, 106 aa, Artificial Sequence
  VL
QSALTQPRSVSGSPGQSVTISCHASQGISNYVAWYQQHPGKAPKLMIYDASNRATGVPDRFSGSKSGNTASLTISGLQAEDEADYYCQQSYSTLLTFGQGTKVEIK

SEQ ID NO: 255 - protein, 106 aa, Artificial Sequence
  VL
QSALTQPRSVSGSPGQSVTISCRVSQSISSYLNWYQQHPGKAPKLMIYDASHRATGVPDRFSGSKSGNTASLTISGLQAEDEADYYCQQYYAPSVTFGQGTKVEIK

SEQ ID NO: 256 - protein, 110 aa, Artificial Sequence
  VL
QSALTQPRSVSGSPGQSVTISCSGSTSNIGSNTVNWYQQHPGKAPKLMIYGKNIRPNGVPDRFSGSKSGNTASLTISGLQAEDEADYYCQSYDSSLSGWVFGGGTKLTVL

SEQ ID NO: 257 - protein, 110 aa, Artificial Sequence
  VL
QSALTQPRSVSGSPGQSVTISCSGSSSNIGSNYVYWYQQHPGKAPKLMIYRNNQRPSGVPDRFSGSKSGNTASLTISGLQAEDEADYYCGTWDSSLSGVVFGGGTKLTVL

SEQ ID NO: 258 - protein, 108 aa, Artificial Sequence
  VL
QSALTQPRSVSGSPGQSVTISCGGNNIGSKSVHWYQQHPGKAPKLMIYDNNKRPSGVPDRFSGSKSGNTASLTISGLQAEDEADYYCAAWDDSLNVPVFGGGTKLTVL

SEQ ID NO: 259 - protein, 111 aa, Artificial Sequence
  VL
QSALTQPRSVSGSPGQSVTISCTGTSSDIGRYNYVSWYQQHPGKAPKLMIYRNTQRPSGVPDRFSGSKSGNTASLTISGLQAEDEADYYCGTWDSSLNAYVFGGGTKLTVL

SEQ ID NO: 260 - protein, 108 aa, Artificial Sequence
  VL
QSALTQPRSVSGSPGQSVTISCSGSNIGNNYVSWYQQHPGKAPKLMIYQDSKRPSGVPDRFSGSKSGNTASLTISGLQAEDEADYYCLVWDRSSDHFVFGGGTKLTVL

SEQ ID NO: 261 - protein, 108 aa, Artificial Sequence
  VL
QSALTQPRSVSGSPGQSVTISCSDNNIGSKSVHWYQQHPGKAPKLMIYDNNKRPSGVPDRFSGSKSGNTASLTISGLQAEDEADYYCGTWDDSLSGPVFGGGTKLTVL

SEQ ID NO: 262 - protein, 107 aa, Artificial Sequence
  VL
SYELTQPPSVSVSPGQTASITCRASQGIGNYLGWYQQKPGQSPVLVIYTASSLQSGIPERFSGSNSGNTATLTISGTQAMDEADYYCQQGYSTPSWTFGQGTKVEIK

SEQ ID NO: 263 - protein, 106 aa, Artificial Sequence
  VL
SYELTQPPSVSVSPGQTASITCHASQGISNYVAWYQQKPGQSPVLVIYDASNRATGIPERFSGSNSGNTATLTISGTQAMDEADYYCQQSYSTLLTFGQGTKVEIK

SEQ ID NO: 264 - protein, 106 aa, Artificial Sequence
  VL
SYELTQPPSVSVSPGQTASITCRVSQSISSYLNWYQQKPGQSPVLVIYDASHRATGIPERFSGSNSGNTATLTISGTQAMDEADYYCQQYYAPSVTFGQGTKVEIK

SEQ ID NO: 265 - protein, 110 aa, Artificial Sequence
  VL
SYELTQPPSVSVSPGQTASITCSGSTSNIGSNTVNWYQQKPGQSPVLVIYGKNIRPNGIPERFSGSNSGNTATLTISGTQAMDEADYYCQSYDSSLSGWVFGGGTKLTVL

SEQ ID NO: 266 - protein, 110 aa, Artificial Sequence
  VL
SYELTQPPSVSVSPGQTASITCSGSSSNIGSNYVYWYQQKPGQSPVLVIYRNNQRPSGIPERFSGSNSGNTATLTISGTQAMDEADYYCGTWDSSLSGVVFGGGTKLTVL

SEQ ID NO: 267 - protein, 108 aa, Artificial Sequence
  VL
SYELTQPPSVSVSPGQTASITCGGNNIGSKSVHWYQQKPGQSPVLVIYDNNKRPSGIPERFSGSNSGNTATLTISGTQAMDEADYYCAAWDDSLNVPVFGGGTKLTVL

SEQ ID NO: 268 - protein, 111 aa, Artificial Sequence
  VL
SYELTQPPSVSVSPGQTASITCTGTSSDIGRYNYVSWYQQKPGQSPVLVIYRNTQRPSGIPERFSGSNSGNTATLTISGTQAMDEADYYCGTWDSSLNAYVFGGGTKLTVL

SEQ ID NO: 269 - protein, 108 aa, Artificial Sequence
  VL
SYELTQPPSVSVSPGQTASITCSGSNIGNNYVSWYQQKPGQSPVLVIYQDSKRPSGIPERFSGSNSGNTATLTISGTQAMDEADYYCLVWDRSSDHFVFGGGTKLTVL

SEQ ID NO: 270 - protein, 108 aa, Artificial Sequence
  VL
SYELTQPPSVSVSPGQTASITCSDNNIGSKSVHWYQQKPGQSPVLVIYDNNKRPSGIPERFSGSNSGNTATLTISGTQAMDEADYYCGTWDDSLSGPVFGGGTKLTVL

SEQ ID NO: 271 - protein, 108 aa, Artificial Sequence
  VL
DIQMTQSPSSLSASVGDRVTITCRASQGIGNYLGWYQQKPGKAPKLLIYTASSLQSGVPSRFSGSGSGTDFTFTISSLQPEDIATYYCQQGYSTPSWTFGQGTKVEIK

SEQ ID NO: 272 - protein, 107 aa, Artificial Sequence
  VL
DIQMTQSPSSLSASVGDRVTITCHASQGISNYVAWYQQKPGKAPKLLIYDASNRATGVPSRFSGSGSGTDFTFTISSLQPEDIATYYCQQSYSTLLTFGQGTKVEIK

SEQ ID NO: 273 - protein, 107 aa, Artificial Sequence
  VL
DIQMTQSPSSLSASVGDRVTITCRVSQSISSYLNWYQQKPGKAPKLLIYDASHRATGVPSRFSGSGSGTDFTFTISSLQPEDIATYYCQQYYAPSVTFGQGTKVEIK

SEQ ID NO: 274 - protein, 111 aa, Artificial Sequence
  VL
DIQMTQSPSSLSASVGDRVTITCSGSTSNIGSNTVNWYQQKPGKAPKLLIYGKNIRPNGVPSRFSGSGSGTDFTFTISSLQPEDIATYYCQSYDSSLSGWVFGGGTKLTVL

SEQ ID NO: 275 - protein, 111 aa, Artificial Sequence
  VL
DIQMTQSPSSLSASVGDRVTITCSGSSSNIGSNYVYWYQQKPGKAPKLLIYRNNQRPSGVPSRFSGSGSGTDFTFTISSLQPEDIATYYCGTWDSSLSGVVFGGGTKLTVL

SEQ ID NO: 276 - protein, 109 aa, Artificial Sequence
  VL
DIQMTQSPSSLSASVGDRVTITCGGNNIGSKSVHWYQQKPGKAPKLLIYDNNKRPSGVPSRFSGSGSGTDFTFTISSLQPEDIATYYCAAWDDSLNVPVFGGGTKLTVL

SEQ ID NO: 277 - protein, 112 aa, Artificial Sequence
  VL
DIQMTQSPSSLSASVGDRVTITCTGTSSDIGRYNYVSWYQQKPGKAPKLLIYRNTQRPSGVPSRFSGSGSGTDFTFTISSLQPEDIATYYCGTWDSSLNAYVFGGGTKLTVL

SEQ ID NO: 278 - protein, 109 aa, Artificial Sequence
  VL
DIQMTQSPSSLSASVGDRVTITCSGSNIGNNYVSWYQQKPGKAPKLLIYQDSKRPSGVPSRFSGSGSGTDFTFTISSLQPEDIATYYCLVWDRSSDHFVFGGGTKLTVL

SEQ ID NO: 279 - protein, 109 aa, Artificial Sequence
  VL
DIQMTQSPSSLSASVGDRVTITCSDNNIGSKSVHWYQQKPGKAPKLLIYDNNKRPSGVPSRFSGSGSGTDFTFTISSLQPEDIATYYCGTWDDSLSGPVFGGGTKLTVL

SEQ ID NO: 280 - protein, 108 aa, Artificial Sequence
  VL
DIQMTQSPSFLSASVGDRVTITCRASQGIGNYLGWYQQKPGKAPKLLIYTASSLQSGVPSRFSGSGSGTDFTLTISSLQPEDFATYYCQQGYSTPSWTFGQGTKVEIK

SEQ ID NO: 281 - protein, 107 aa, Artificial Sequence
  VL
DIQMTQSPSFLSASVGDRVTITCHASQGISNYVAWYQQKPGKAPKLLIYDASNRATGVPSRFSGSGSGTDFTLTISSLQPEDFATYYCQQSYSTLLTFGQGTKVEIK

SEQ ID NO: 282 - protein, 107 aa, Artificial Sequence
  VL
DIQMTQSPSFLSASVGDRVTITCRVSQSISSYLNWYQQKPGKAPKLLIYDASHRATGVPSRFSGSGSGTDFTLTISSLQPEDFATYYCQQYYAPSVTFGQGTKVEIK

SEQ ID NO: 283 - protein, 111 aa, Artificial Sequence
  VL
DIQMTQSPSFLSASVGDRVTITCSGSTSNIGSNTVNWYQQKPGKAPKLLIYGKNIRPNGVPSRFSGSGSGTDFTLTISSLQPEDFATYYCQSYDSSLSGWVFGGGTKLTVL

SEQ ID NO: 284 - protein, 111 aa, Artificial Sequence
  VL
DIQMTQSPSFLSASVGDRVTITCSGSSSNIGSNYVYWYQQKPGKAPKLLIYRNNQRPSGVPSRFSGSGSGTDFTLTISSLQPEDFATYYCGTWDSSLSGVVFGGGTKLTVL

SEQ ID NO: 285 - protein, 109 aa, Artificial Sequence
  VL
DIQMTQSPSFLSASVGDRVTITCGGNNIGSKSVHWYQQKPGKAPKLLIYDNNKRPSGVPSRFSGSGSGTDFTLTISSLQPEDFATYYCAAWDDSLNVPVFGGGTKLTVL

SEQ ID NO: 286 - protein, 112 aa, Artificial Sequence
  VL
DIQMTQSPSFLSASVGDRVTITCTGTSSDIGRYNYVSWYQQKPGKAPKLLIYRNTQRPSGVPSRFSGSGSGTDFTLTISSLQPEDFATYYCGTWDSSLNAYVFGGGTKLTVL

SEQ ID NO: 287 - protein, 109 aa, Artificial Sequence
  VL
DIQMTQSPSFLSASVGDRVTITCSGSNIGNNYVSWYQQKPGKAPKLLIYQDSKRPSGVPSRFSGSGSGTDFTLTISSLQPEDFATYYCLVWDRSSDHFVFGGGTKLTVL

SEQ ID NO: 288 - protein, 109 aa, Artificial Sequence
  VL
DIQMTQSPSFLSASVGDRVTITCSDNNIGSKSVHWYQQKPGKAPKLLIYDNNKRPSGVPSRFSGSGSGTDFTLTISSLQPEDFATYYCGTWDDSLSGPVFGGGTKLTVL

SEQ ID NO: 289 - protein, 108 aa, Artificial Sequence
  VL
DIVMTQSPLSLPVTPGEPASISCRASQGIGNYLGWYLQKPGQSPQLLIYTASSLQSGVPDRFSGSGSGTDFTLKISRVEAEDVGVYYCQQGYSTPSWTFGQGTKVEIK

SEQ ID NO: 290 - protein, 107 aa, Artificial Sequence
  VL
DIVMTQSPLSLPVTPGEPASISCHASQGISNYVAWYLQKPGQSPQLLIYDASNRATGVPDRFSGSGSGTDFTLKISRVEAEDVGVYYCQQSYSTLLTFGQGTKVEIK

SEQ ID NO: 291 - protein, 107 aa, Artificial Sequence
  VL
DIVMTQSPLSLPVTPGEPASISCRVSQSISSYLNWYLQKPGQSPQLLIYDASHRATGVPDRFSGSGSGTDFTLKISRVEAEDVGVYYCQQYYAPSVTFGQGTKVEIK

SEQ ID NO: 292 - protein, 111 aa, Artificial Sequence
  VL
DIVMTQSPLSLPVTPGEPASISCSGSTSNIGSNTVNWYLQKPGQSPQLLIYGKNIRPNGVPDRFSGSGSGTDFTLKISRVEAEDVGVYYCQSYDSSLSGWVFGGGTKLTVL

SEQ ID NO: 293 - protein, 111 aa, Artificial Sequence
  VL
DIVMTQSPLSLPVTPGEPASISCSGSSSNIGSNYVYWYLQKPGQSPQLLIYRNNQRPSGVPDRFSGSGSGTDFTLKISRVEAEDVGVYYCGTWDSSLSGVVFGGGTKLTVL

SEQ ID NO: 294 - protein, 109 aa, Artificial Sequence
  VL
DIVMTQSPLSLPVTPGEPASISCGGNNIGSKSVHWYLQKPGQSPQLLIYDNNKRPSGVPDRFSGSGSGTDFTLKISRVEAEDVGVYYCAAWDDSLNVPVFGGGTKLTVL

SEQ ID NO: 295 - protein, 112 aa, Artificial Sequence
  VL
DIVMTQSPLSLPVTPGEPASISCTGTSSDIGRYNYVSWYLQKPGQSPQLLIYRNTQRPSGVPDRFSGSGSGTDFTLKISRVEAEDVGVYYCGTWDSSLNAYVFGGGTKLTVL

SEQ ID NO: 296 - protein, 109 aa, Artificial Sequence
  VL
DIVMTQSPLSLPVTPGEPASISCSGSNIGNNYVSWYLQKPGQSPQLLIYQDSKRPSGVPDRFSGSGSGTDFTLKISRVEAEDVGVYYCLVWDRSSDHFVFGGGTKLTVL

SEQ ID NO: 297 - protein, 109 aa, Artificial Sequence
  VL
DIVMTQSPLSLPVTPGEPASISCSDNNIGSKSVHWYLQKPGQSPQLLIYDNNKRPSGVPDRFSGSGSGTDFTLKISRVEAEDVGVYYCGTWDDSLSGPVFGGGTKLTVL

SEQ ID NO: 298 - protein, 108 aa, Artificial Sequence
  VL
EIVMTQSPATLSVSPGERATLSCRASQGIGNYLGWYQQKPGQAPRLLIYTASSLQSGIPARFSGSGSGTEFTLTISSLQSEDFAVYYCQQGYSTPSWTFGQGTKVEIK

SEQ ID NO: 299 - protein, 107 aa, Artificial Sequence
  VL
EIVMTQSPATLSVSPGERATLSCHASQGISNYVAWYQQKPGQAPRLLIYDASNRATGIPARFSGSGSGTEFTLTISSLQSEDFAVYYCQQSYSTLLTFGQGTKVEIK

SEQ ID NO: 300 - protein, 107 aa, Artificial Sequence
  VL
EIVMTQSPATLSVSPGERATLSCRVSQSISSYLNWYQQKPGQAPRLLIYDASHRATGIPARFSGSGSGTEFTLTISSLQSEDFAVYYCQQYYAPSVTFGQGTKVEIK

SEQ ID NO: 301 - protein, 111 aa, Artificial Sequence
  VL
EIVMTQSPATLSVSPGERATLSCSGSTSNIGSNTVNWYQQKPGQAPRLLIYGKNIRPNGIPARFSGSGSGTEFTLTISSLQSEDFAVYYCQSYDSSLSGWVFGGGTKLTVL

SEQ ID NO: 302 - protein, 111 aa, Artificial Sequence
  VL
EIVMTQSPATLSVSPGERATLSCSGSSSNIGSNYVYWYQQKPGQAPRLLIYRNNQRPSGIPARFSGSGSGTEFTLTISSLQSEDFAVYYCGTWDSSLSGVVFGGGTKLTVL

SEQ ID NO: 303 - protein, 109 aa, Artificial Sequence
  VL
EIVMTQSPATLSVSPGERATLSCGGNNIGSKSVHWYQQKPGQAPRLLIYDNNKRPSGIPARFSGSGSGTEFTLTISSLQSEDFAVYYCAAWDDSLNVPVFGGGTKLTVL

SEQ ID NO: 304 - protein, 112 aa, Artificial Sequence
  VL
EIVMTQSPATLSVSPGERATLSCTGTSSDIGRYNYVSWYQQKPGQAPRLLIYRNTQRPSGIPARFSGSGSGTEFTLTISSLQSEDFAVYYCGTWDSSLNAYVFGGGTKLTVL

SEQ ID NO: 305 - protein, 109 aa, Artificial Sequence
  VL
EIVMTQSPATLSVSPGERATLSCSGSNIGNNYVSWYQQKPGQAPRLLIYQDSKRPSGIPARFSGSGSGTEFTLTISSLQSEDFAVYYCLVWDRSSDHFVFGGGTKLTVL

SEQ ID NO: 306 - protein, 109 aa, Artificial Sequence
  VL
EIVMTQSPATLSVSPGERATLSCSDNNIGSKSVHWYQQKPGQAPRLLIYDNNKRPSGIPARFSGSGSGTEFTLTISSLQSEDFAVYYCGTWDDSLSGPVFGGGTKLTVL

SEQ ID NO: 307 - protein, 108 aa, Artificial Sequence
  VL
EIVLTQSPGTLSLSPGERATLSCRASQGIGNYLGWYQQKPGQAPRLLIYTASSLQSGIPDRFSGSGSGTDFTLTISRLEPEDFAVYYCQQGYSTPSWTFGQGTKVEIK

SEQ ID NO: 308 - protein, 107 aa, Artificial Sequence
  VL
EIVLTQSPGTLSLSPGERATLSCHASQGISNYVAWYQQKPGQAPRLLIYDASNRATGIPDRFSGSGSGTDFTLTISRLEPEDFAVYYCQQSYSTLLTFGQGTKVEIK

SEQ ID NO: 309 - protein, 107 aa, Artificial Sequence
  VL
EIVLTQSPGTLSLSPGERATLSCRVSQSISSYLNWYQQKPGQAPRLLIYDASHRATGIPDRFSGSGSGTDFTLTISRLEPEDFAVYYCQQYYAPSVTFGQGTKVEIK

SEQ ID NO: 310 - protein, 111 aa, Artificial Sequence
  VL
EIVLTQSPGTLSLSPGERATLSCSGSTSNIGSNTVNWYQQKPGQAPRLLIYGKNIRPNGIPDRFSGSGSGTDFTLTISRLEPEDFAVYYCQSYDSSLSGWVFGGGTKLTVL

SEQ ID NO: 311 - protein, 111 aa, Artificial Sequence
  VL
EIVLTQSPGTLSLSPGERATLSCSGSSSNIGSNYVYWYQQKPGQAPRLLIYRNNQRPSGIPDRFSGSGSGTDFTLTISRLEPEDFAVYYCGTWDSSLSGVVFGGGTKLTVL

SEQ ID NO: 312 - protein, 109 aa, Artificial Sequence
  VL
EIVLTQSPGTLSLSPGERATLSCGGNNIGSKSVHWYQQKPGQAPRLLIYDNNKRPSGIPDRFSGSGSGTDFTLTISRLEPEDFAVYYCAAWDDSLNVPVFGGGTKLTVL

SEQ ID NO: 313 - protein, 112 aa, Artificial Sequence
  VL
EIVLTQSPGTLSLSPGERATLSCTGTSSDIGRYNYVSWYQQKPGQAPRLLIYRNTQRPSGIPDRFSGSGSGTDFTLTISRLEPEDFAVYYCGTWDSSLNAYVFGGGTKLTVL

SEQ ID NO: 314 - protein, 109 aa, Artificial Sequence
  VL
EIVLTQSPGTLSLSPGERATLSCSGSNIGNNYVSWYQQKPGQAPRLLIYQDSKRPSGIPDRFSGSGSGTDFTLTISRLEPEDFAVYYCLVWDRSSDHFVFGGGTKLTVL

SEQ ID NO: 315 - protein, 109 aa, Artificial Sequence
  VL
EIVLTQSPGTLSLSPGERATLSCSDNNIGSKSVHWYQQKPGQAPRLLIYDNNKRPSGIPDRFSGSGSGTDFTLTISRLEPEDFAVYYCGTWDDSLSGPVFGGGTKLTVL

SEQ ID NO: 316 - protein, 108 aa, Artificial Sequence
  VL
EIVLTQSPGTLSLSPGERATLSCRASQGIGNYLGWYQQKPGQAPRLLIYTASSLQSGIPDRFSGSGSGTDFTLTISHLEPEDFAVYYCQQGYSTPSWTFGQGTKVEIK

SEQ ID NO: 317 - protein, 107 aa, Artificial Sequence
  VL
EIVLTQSPGTLSLSPGERATLSCHASQGISNYVAWYQQKPGQAPRLLIYDASNRATGIPDRFSGSGSGTDFTLTISHLEPEDFAVYYCQQSYSTLLTFGQGTKVEIK

SEQ ID NO: 318 - protein, 107 aa, Artificial Sequence
  VL
EIVLTQSPGTLSLSPGERATLSCRVSQSISSYLNWYQQKPGQAPRLLIYDASHRATGIPDRFSGSGSGTDFTLTISHLEPEDFAVYYCQQYYAPSVTFGQGTKVEIK

SEQ ID NO: 319 - protein, 111 aa, Artificial Sequence
  VL
EIVLTQSPGTLSLSPGERATLSCSGSTSNIGSNTVNWYQQKPGQAPRLLIYGKNIRPNGIPDRFSGSGSGTDFTLTISHLEPEDFAVYYCQSYDSSLSGWVFGGGTKLTVL

SEQ ID NO: 320 - protein, 111 aa, Artificial Sequence
  VL
EIVLTQSPGTLSLSPGERATLSCSGSSSNIGSNYVYWYQQKPGQAPRLLIYRNNQRPSGIPDRFSGSGSGTDFTLTISHLEPEDFAVYYCGTWDSSLSGVVFGGGTKLTVL

SEQ ID NO: 321 - protein, 109 aa, Artificial Sequence
  VL
EIVLTQSPGTLSLSPGERATLSCGGNNIGSKSVHWYQQKPGQAPRLLIYDNNKRPSGIPDRFSGSGSGTDFTLTISHLEPEDFAVYYCAAWDDSLNVPVFGGGTKLTVL

SEQ ID NO: 322 - protein, 112 aa, Artificial Sequence
  VL
EIVLTQSPGTLSLSPGERATLSCTGTSSDIGRYNYVSWYQQKPGQAPRLLIYRNTQRPSGIPDRFSGSGSGTDFTLTISHLEPEDFAVYYCGTWDSSLNAYVFGGGTKLTVL

SEQ ID NO: 323 - protein, 109 aa, Artificial Sequence
  VL
EIVLTQSPGTLSLSPGERATLSCSGSNIGNNYVSWYQQKPGQAPRLLIYQDSKRPSGIPDRFSGSGSGTDFTLTISHLEPEDFAVYYCLVWDRSSDHFVFGGGTKLTVL

SEQ ID NO: 324 - protein, 109 aa, Artificial Sequence
  VL
EIVLTQSPGTLSLSPGERATLSCSDNNIGSKSVHWYQQKPGQAPRLLIYDNNKRPSGIPDRFSGSGSGTDFTLTISHLEPEDFAVYYCGTWDDSLSGPVFGGGTKLTVL

SEQ ID NO: 325 - protein, 108 aa, Artificial Sequence
  VL
DIVMTQSPDSLAVSLGERATINCRASQGIGNYLGWYQQKPGQPPKLLIYTASSLQSGVPDRFSGSGSGTDFTLTISSLQAEDVAVYYCQQGYSTPSWTFGQGTKVEIK

SEQ ID NO: 326 - protein, 107 aa, Artificial Sequence
  VL
DIVMTQSPDSLAVSLGERATINCHASQGISNYVAWYQQKPGQPPKLLIYDASNRATGVPDRFSGSGSGTDFTLTISSLQAEDVAVYYCQQSYSTLLTFGQGTKVEIK

SEQ ID NO: 327 - protein, 107 aa, Artificial Sequence
  VL
DIVMTQSPDSLAVSLGERATINCRVSQSISSYLNWYQQKPGQPPKLLIYDASHRATGVPDRFSGSGSGTDFTLTISSLQAEDVAVYYCQQYYAPSVTFGQGTKVEIK

SEQ ID NO: 328 - protein, 111 aa, Artificial Sequence
  VL
DIVMTQSPDSLAVSLGERATINCSGSTSNIGSNTVNWYQQKPGQPPKLLIYGKNIRPNGVPDRFSGSGSGTDFTLTISSLQAEDVAVYYCQSYDSSLSGWVFGGGTKLTVL

SEQ ID NO: 329 - protein, 111 aa, Artificial Sequence
  VL
DIVMTQSPDSLAVSLGERATINCSGSSSNIGSNYVYWYQQKPGQPPKLLIYRNNQRPSGVPDRFSGSGSGTDFTLTISSLQAEDVAVYYCGTWDSSLSGVVFGGGTKLTVL

SEQ ID NO: 330 - protein, 109 aa, Artificial Sequence
  VL
DIVMTQSPDSLAVSLGERATINCGGNNIGSKSVHWYQQKPGQPPKLLIYDNNKRPSGVPDRFSGSGSGTDFTLTISSLQAEDVAVYYCAAWDDSLNVPVFGGGTKLTVL

SEQ ID NO: 331 - protein, 112 aa, Artificial Sequence
  VL
DIVMTQSPDSLAVSLGERATINCTGTSSDIGRYNYVSWYQQKPGQPPKLLIYRNTQRPSGVPDRFSGSGSGTDFTLTISSLQAEDVAVYYCGTWDSSLNAYVFGGGTKLTVL

SEQ ID NO: 332 - protein, 109 aa, Artificial Sequence
  VL
DIVMTQSPDSLAVSLGERATINCSGSNIGNNYVSWYQQKPGQPPKLLIYQDSKRPSGVPDRFSGSGSGTDFTLTISSLQAEDVAVYYCLVWDRSSDHFVFGGGTKLTVL

SEQ ID NO: 333 - protein, 109 aa, Artificial Sequence
  VL
DIVMTQSPDSLAVSLGERATINCSDNNIGSKSVHWYQQKPGQPPKLLIYDNNKRPSGVPDRFSGSGSGTDFTLTISSLQAEDVAVYYCGTWDDSLSGPVFGGGTKLTVL

SEQ ID NO: 334 - protein, 11 aa, Artificial Sequence
  VH_FR4
WGQGTLVTVSS

SEQ ID NO: 335 - protein, 10 aa, Artificial Sequence
  VL_FR4
FGQGTKVEIK

SEQ ID NO: 336 - protein, 10 aa, Artificial Sequence
  VL_FR4
FGGGTKLTVL